(19) United States
(12) Patent Application Publication
(10) Pub. No.: US 2025/0235411 A1
(43) Pub. Date: Jul. 24, 2025

(54) COMPOSITIONS AND METHODS FOR DELIVERY OF AGENTS

(71) Applicant: Kernal Biologics, Inc., Cambridge, MA (US)

(72) Inventors: Thomas Colace, Arlington, MA (US); Yusuf Erkul, Cambridge, MA (US); Jieni Xu, Cambridge, MA (US); Burak Yilmaz, Cambridge, MA (US)

(21) Appl. No.: 18/700,942

(22) PCT Filed: Oct. 14, 2022
(86) PCT No.: PCT/US2022/046785

§ 371 Date: Apr. 12, 2024

Related U.S. Application Data
(60) Provisional application No. 63/255,871, filed on Oct. 14, 2021.

(51) Int. Cl. A61K 9/51; A61K 31/7105; A61K 47/68; A61K 47/69; A61K 48/00; C12Q 1/6883

(52) U.S. Cl. CPC A61K 9/5123; A61K 31/7105; A61K 47/6849; A61K 47/6889; A61K 47/6929; A61K 48/0033; C12Q 1/6883; C12Q 2600/158; C12Q 2600/178

(57) ABSTRACT

The present disclosure provides lipid nanoparticle compositions and methods of use. Among other things the present disclosure provides lipid nanoparticle compositions which increased specificity for specific cells or tissues. The present disclosure provides methods of use of the disclosed lipid nanoparticles.

### BACKGROUND

The targeted delivery of nucleic acids represents a continuing medical challenge. Attempts have been made to use lipid nanoparticles as delivery vehicles for biologically active substances. There remains a poor understanding of which compositions and methods of lipid nanoparticles provide specific tissue targeted delivery.

### SUMMARY

The present disclosure recognizes a need for compositions, preparations, and methods of use of nanoparticles for the targeted delivery of nucleic acids. Among other things, the present disclosure recognizes that specific compositions, preparations, and methods of use of nanoparticles are useful for the targeted delivery of nucleic acids to particular tissues in vivo. In some embodiments, the present disclosure provides compositions and methods of use of nanoparticles for the delivery of nucleic acids to lung tissue.

In some embodiments, the present disclosure provides a nucleic acid lipid particle composition comprising: a nucleic acid; and lipid components including:

| Component | Range |
|---|---|
| Cationic Ionizable Lipid pKa < 7 | 0-60% |
| Cationic Ionizable Lipid pKa > 7 | 0-80% |
| Sterol | 0-40% |
| Helper Lipid | 0-20% |
| PEGylated Lipid | 0-25% |
| PEG-Ligand Lipid | 0-25% |

In some embodiments, the present disclosure provides a nucleic acid lipid particle composition comprising: a nucleic acid; and lipid components including:

| Component | Range |
|---|---|
| Cationic Ionizable Lipid pKa < 7 | 30-60% |
| Sterol | 20-40% |
| Helper Lipid | 10-30% |
| PEGylated Lipid | 0-5% |
| PEG-Ligand | 0-5% |

In some embodiments, the present disclosure provides methods and compositions for treating disease using a nucleic acid lipid particle described herein. In some embodiments, a disease treated by methods or compositions described herein is a cancer, a lung disease, a disease effecting a B-Cell. In some embodiments, the present disclosure provides methods and compositions for delivering a nucleic acid to a tissue of a subject. In some embodiments, a tissue is extrahepatic. In some embodiments, a tissue is or comprises a lung tissue. In some embodiments, a tissue is or comprises a B-cell.

In some embodiments, the present disclosure provides methods and compositions for treating diseases of the lung or delivering a nucleic acid to a tissue that is or comprises lung tissue the composition selected from selected from the group consisting of:

| Formulation | Composition |
|---|---|
| K056 | 50 mol % DOTAP, 25 mol % LIP001; |
| | 18.75% cholesterol; 5 mol % DSPC; 0.75 |
| | mol % PEG-DMG, 0.5% PEG-ligand |
| K057 | 50 mol % DOTAP, 25 mol % LIP001; |
| | 19.15% cholesterol; 5 mol % DSPC; 0.75 |
| | mol % PEG-DMG, 0.1% PEG-ligand |
| K058 | 50 mol % DOTAP, 25 mol % LIP001; |
| | 19.2% cholesterol; 5 mol % DSPC; 0.75 |
| | mol % PEG-DMG, 0.05% PEG-ligand |
| K099: | 50 mol % DOTAP, 25 mol % LIP003; |
| | 18.5% cholesterol; 5 mol % DSPC; 0.5 |
| | mol % PEG-DMG, 1% PEG-ligand |
| K100 | 48.2 mol % DOTAP, 24.1 mol % LIP003; |
| | 17.8% cholesterol; 4.8 mol % DSPC; 4 |
| | mol % PEG-DMG, 1% PEG-ligand |
| K101 | 45.7 mol % DOTAP, 22.8 mol % LIP003; |
| | 16.9% cholesterol; 4.6 mol % DSPC; 9 |
| | mol % PEG-DMG, 1% PEG-ligand |
| K102 | 43.1 mol % DOTAP, 21.6 mol % LIP003; |
| | 16% cholesterol; 4.3 mol % DSPC; 14 |
| | mol % PEG-DMG, 1% PEG-ligand |
| K261 | 50 mol % LIP019; 25 mol % LIP003; |
| | 19.25% cholesterol; 5 mol % DSPC; 0.75 |
| | mol % PEG-DMG. |
| K263 | 50 mol % LIP021; 25 mol % LIP003; |
| | 19.25% cholesterol; 5 mol % DSPC; 0.75 |
| | mol % PEG-DMG |
| K269 | 50 mol % LIP021; 25 mol % LIP001; |
| | 19.25% cholesterol; 5 mol % DSPC; 0.75 |
| | mol % PEG-DMG |

In some embodiments, the present disclosure provides methods and compositions for delivering a nucleic acid to extrahepatic tissue selected from the group consisting of:

| Formulation | Composition |
|---|---|
| K029 | 50 mol % LIP002; 27.5% cholesterol; 22 |
| | mol % DSPC; 0.5 mol % PEG-DMG. |
| K030 | 50 mol % LIP003; 27.5% cholesterol; 22 |
| | mol % DSPC; 0.5 mol % PEG-DMG |
| K108 | 50 mol % LIP003; 27.5% cholesterol; 22 |
| | mol % DSPC; 0.5 mol % PEG-DSPE |
| K111 | 50 mol % LIP003; 25% cholesterol; 22 |
| | mol % DSPC; 3 mol % FA-PEG-DSPE |
| K051 | 46.8 mol % LIP003; 36.8% cholesterol; 15.9 |
| | mol % DSPC; 0.5 mol % PEG-DMG |
| K052 | 46.8 mol % LIP003; 24.9% cholesterol; 27.9 |
| | mol % DSPC; 0.5 mol % PEG-DMG |
| K053 | 34.8 mol % LIP003; 36.8% cholesterol; 27.9 |
| | mol % DSPC; 0.5 mol % PEG-DMG |
| K054 | 42.8 mol % LIP003; 32.8% cholesterol; 23.9 |
| | mol % DSPC; 0.5 mol % PEG-DMG |

In some embodiments, the present disclosure provides methods and compositions for functionalizing LNPs. In some embodiments, an LNP is functionalized by conjugation of a targeting entity to the surface of an LNP. In some embodiments, a targeting entity is an antibody or antibody fragment. In some embodiments, a targeting entity is an antibody or antibody fragment that binds or targets CD19.

In some embodiments, the present disclosure provides methods and compositions for delivering a nucleic acid to B cells. In some embodiments, the present disclosure provides methods and compositions for delivering a nucleic acid to B cells selected from the group consisting of:

| Formulation | Composition |
|---|---|
| K258 (DTT) | 50 mol % LIP003; 27.5% cholesterol; 22 |
| | mol % DSPC; 0.5 mol % Maleimide-PEG- |
| | DSPE, RG7/Maleimide molar ratio = 0.32/1, |
| | CD19 antibody/RG7 weight ratio = 0.1/1 |
| K259 (DTT) | 50 mol % LIP003; 27.5% cholesterol; 22 |
| | mol % DSPC; 0.5 mol % Maleimide-PEG- |
| | DSPE, RG7/Maleimide molar ratio = 0.32/1, |
| | CD19 antibody/RG7 weight ratio = 1/1 |
| K305 (DTT) | 50 mol % LIP003; 27.5% cholesterol; 22 |
| | mol % DSPC; 0.5 mol % Maleimide-PEG- |
| | DSPE, RG7/Maleimide molar ratio = 0.32/1, |
| | CD19 antibody/RG7 molar ratio = 0.5/1 |
| K306 (DTT) | 50 mol % LIP003; 27.5% cholesterol; 22 |
| | mol % DSPC; 0.5 mol % Maleimide-PEG- |
| | DSPE, RG7/Maleimide molar ratio-0.32/1, |
| | CD19 antibody/RG7 molar ratio = 0.5/1 |
| K361 (DTT) | 50 mol % LIP003; 27.5% cholesterol; 22 |
| | mol % DSPC; 0.5 mol % Maleimide-PEG- |
| | DSPE, RG7/Maleimide molar ratio = 0.16/1, |
| | CD19 antibody/RG7 molar ratio = 0.1/1 |
| K362 (DTT) | 50 mol % LIP003; 27.5% cholesterol; 22 |
| | mol % DSPC; 0.5 mol % Maleimide-PEG- |
| | DSPE, RG7/Maleimide molar ratio = 0.06/1, |
| | CD19 antibody/RG7 molar ratio-0.5/1 |
| K305 (SATA) | 50 mol % LIP003; 27.5% cholesterol; 22 |
| | mol % DSPC; 0.5 mol % Maleimide-PEG- |
| | DSPE, RG7/Maleimide molar ratio = 0.02/1, |
| | CD19 antibody/RG7 molar ratio-0.5/1 |
| K306 (SATA) | 50 mol % LIP003; 27.5% cholesterol; 22 |
| | mol % DSPC; 0.5 mol % Maleimide-PEG- |
| | DSPE, RG7/Maleimide molar ratio = 0.02/1, |
| | CD19 antibody/RG7 molar ratio-0.5/1 |
| K357 (DBCO/azide) | 50 mol % LIP003; 27.5% cholesterol; 22 |
| | mol % DSPC; 0.5 mol % Maleimide-PEG- |
| | DSPE, RG7/Maleimide molar ratio = 0.26/1, |
| | CD19 antibody/RG7 molar ratio = 0.5/1 |
| K358 (DBCO/Azide) | 50 mol % LIP003; 27.5% cholesterol; 22 |
| | mol % DSPC; 0.5 mol % Maleimide-PEG- |
| | DSPE, RG7/Maleimide molar ratio = 0.13/1, |
| | CD19 antibody/RG7 molar ratio = 0.5/1 |

### BRIEF DESCRIPTION OF THE DRAWING

FIG. 1 shows in vivo bioluminesence images for the four (LNP1; LNP2; LNP3 and LNP4) lipid nanoparticles (LNPs) tested.

FIG. 2 shows ex vivo luminescence of indicated tissues for each of the four LNPs tested.

FIG. 3 shows quantitation of ex vivo luminescence of indicated tissues for each of the four LNPs tested.

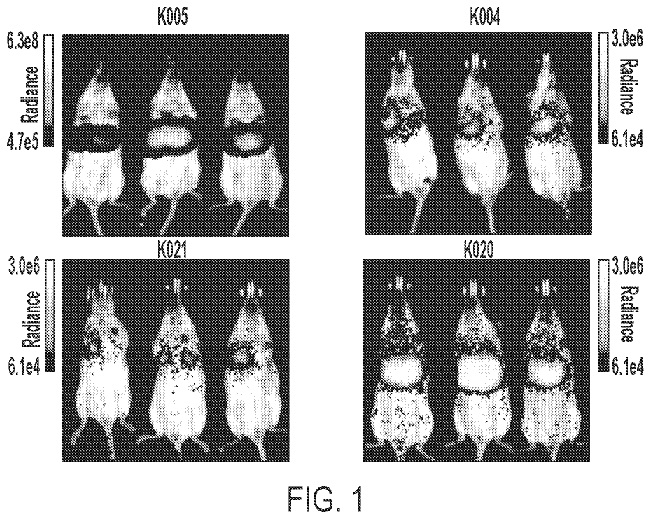

FIG. 1 shows ex vivo lung luminescence for the indicated LNPs 24 hrs post dosing.

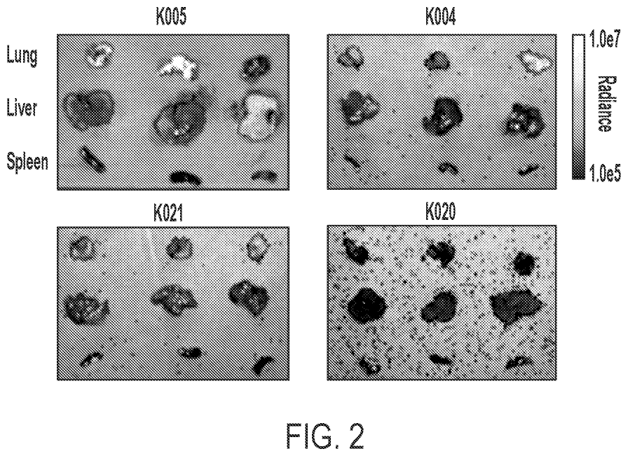

FIG. 2 shows the percentage of luminescence attributable to the lung tissue ex vivo.

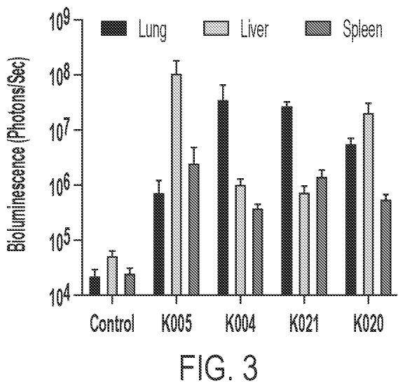

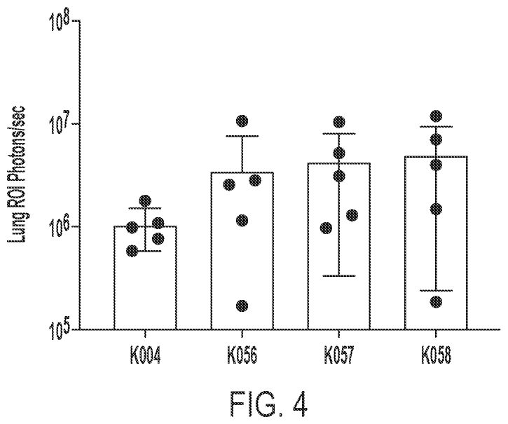

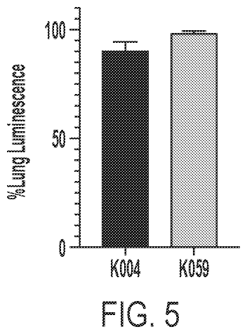

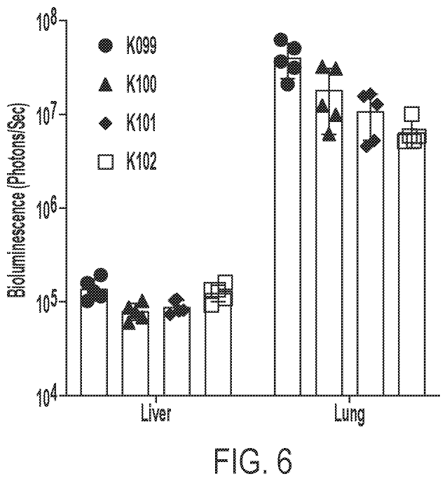

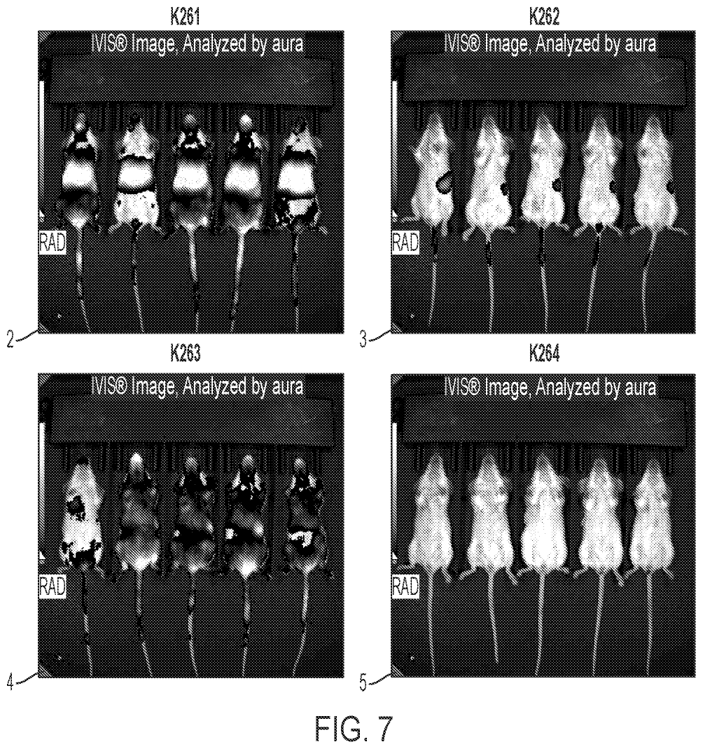

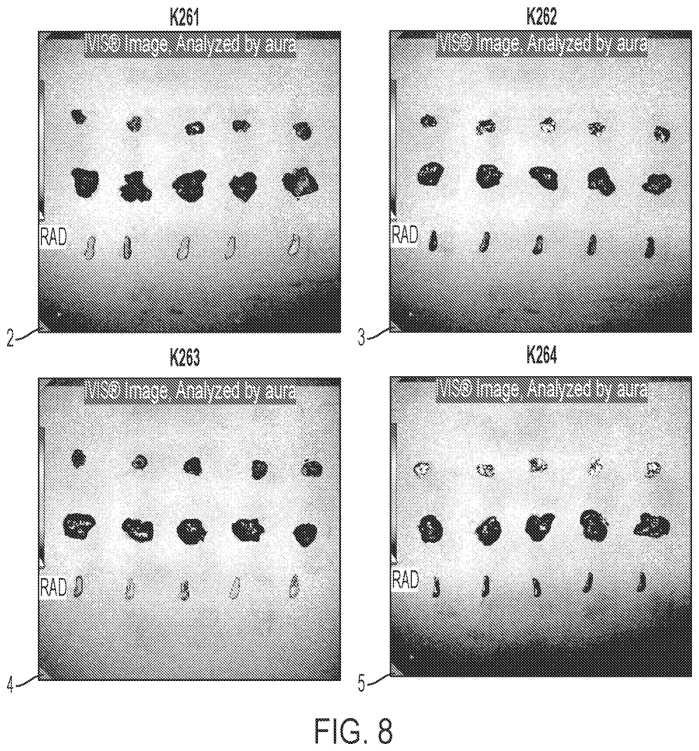

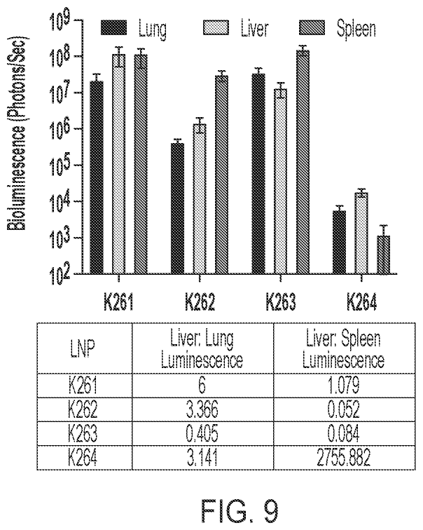

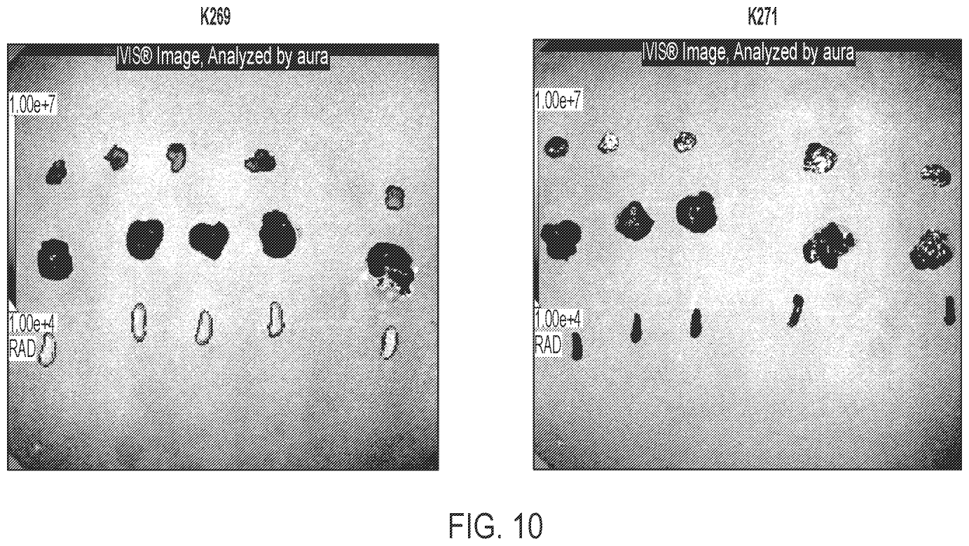

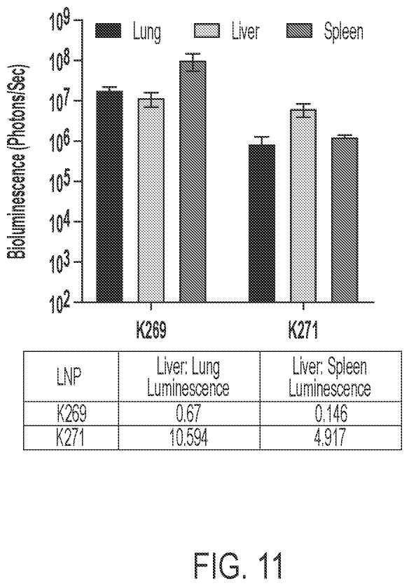

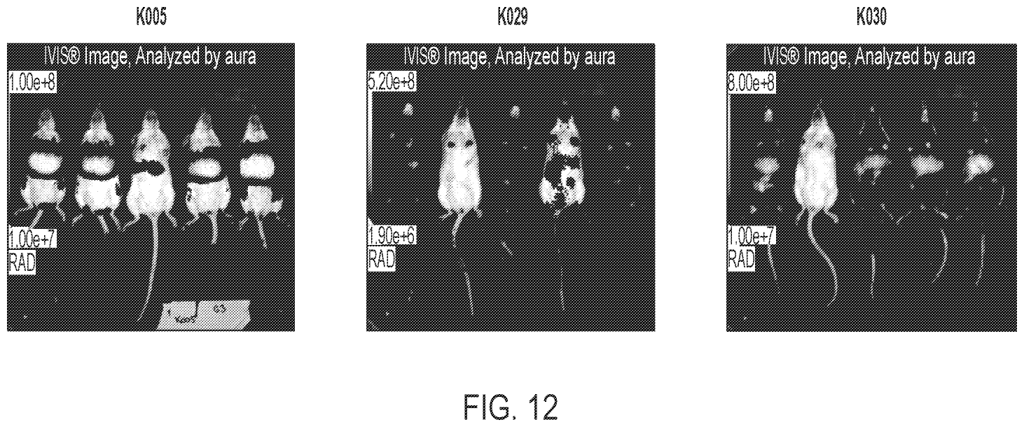

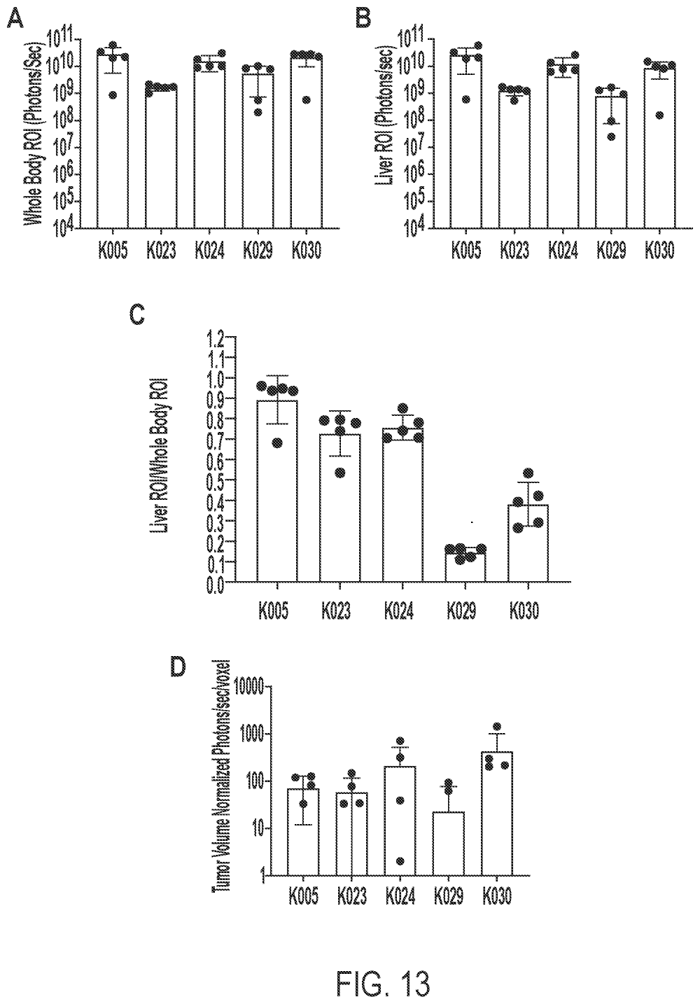

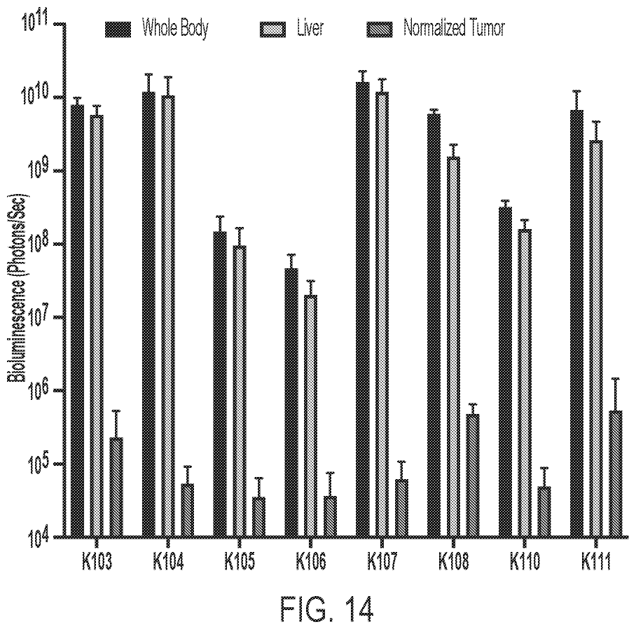

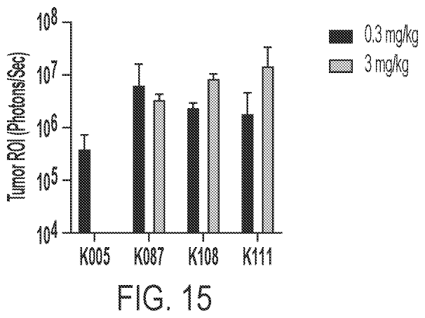

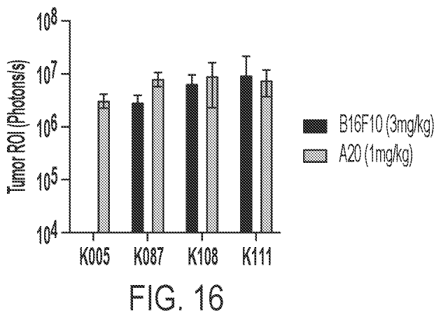

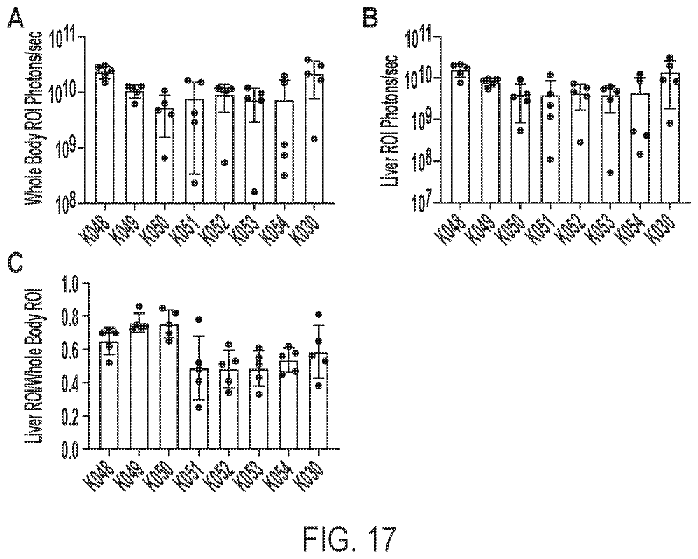

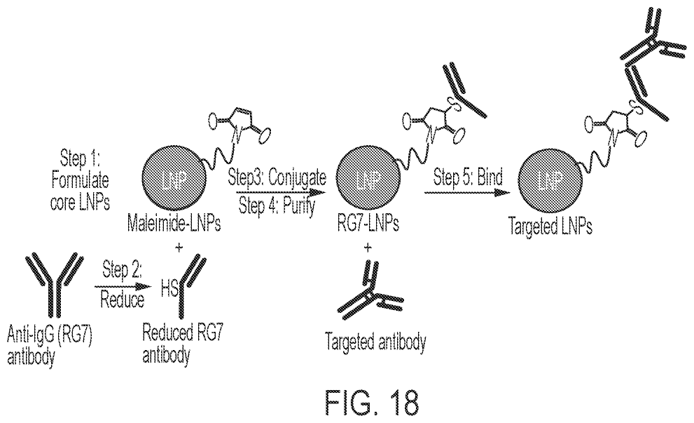

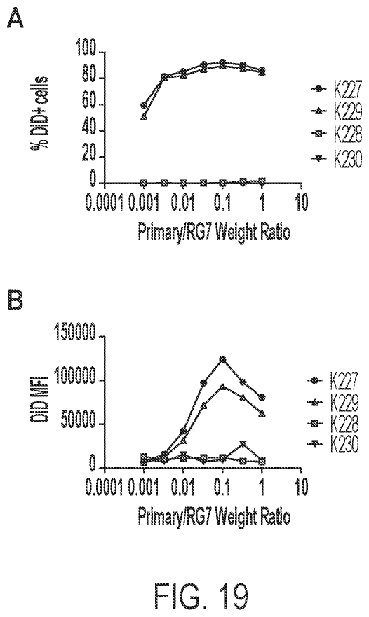

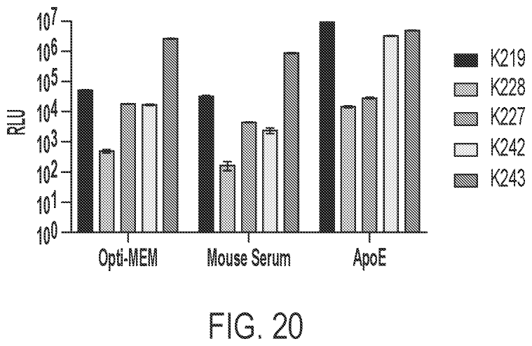

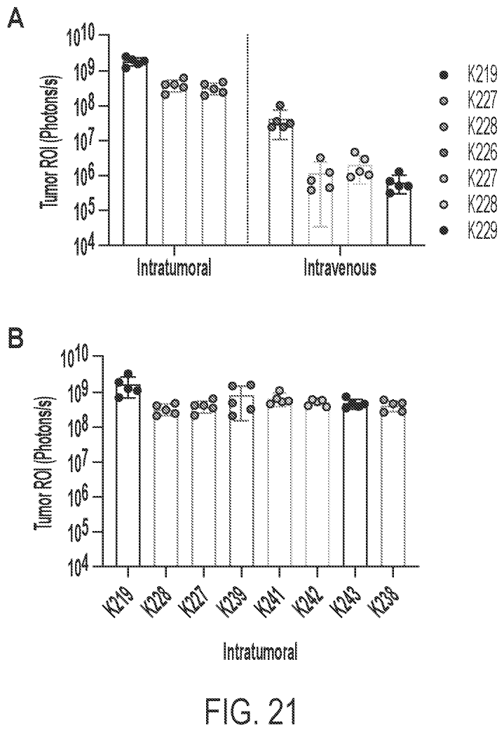

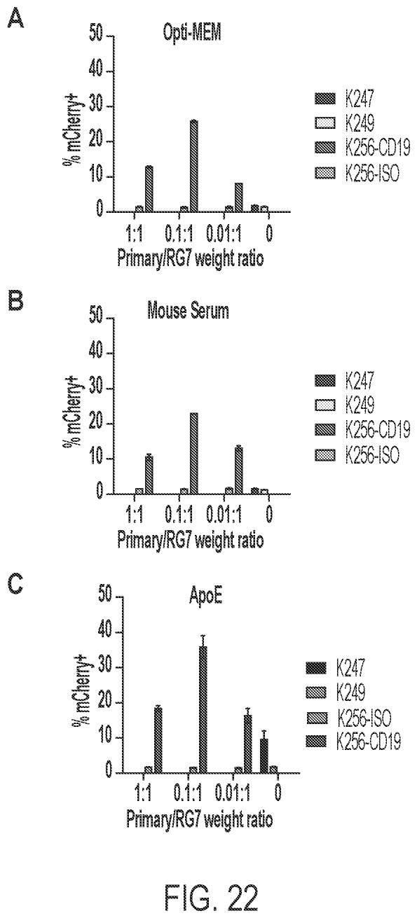

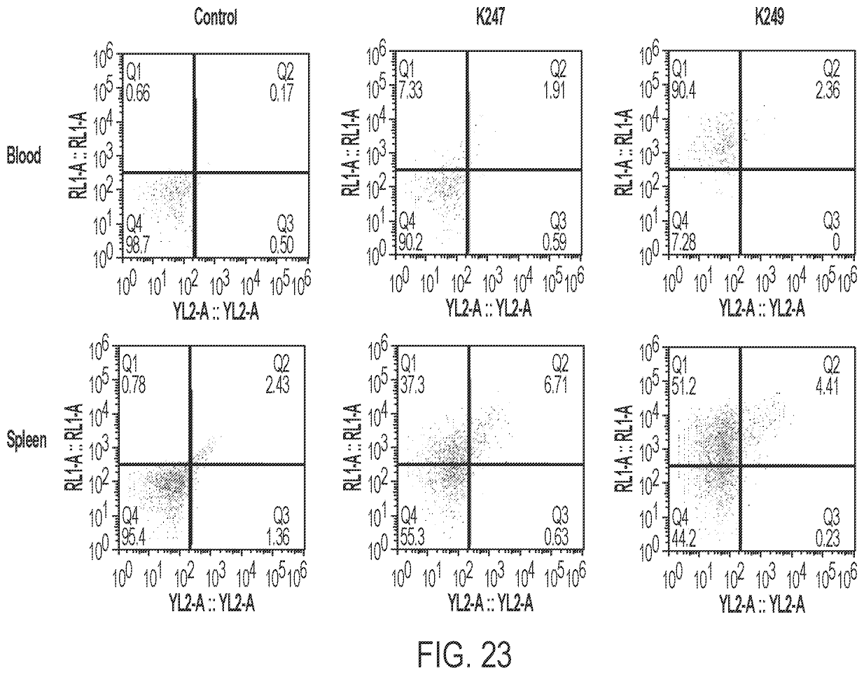

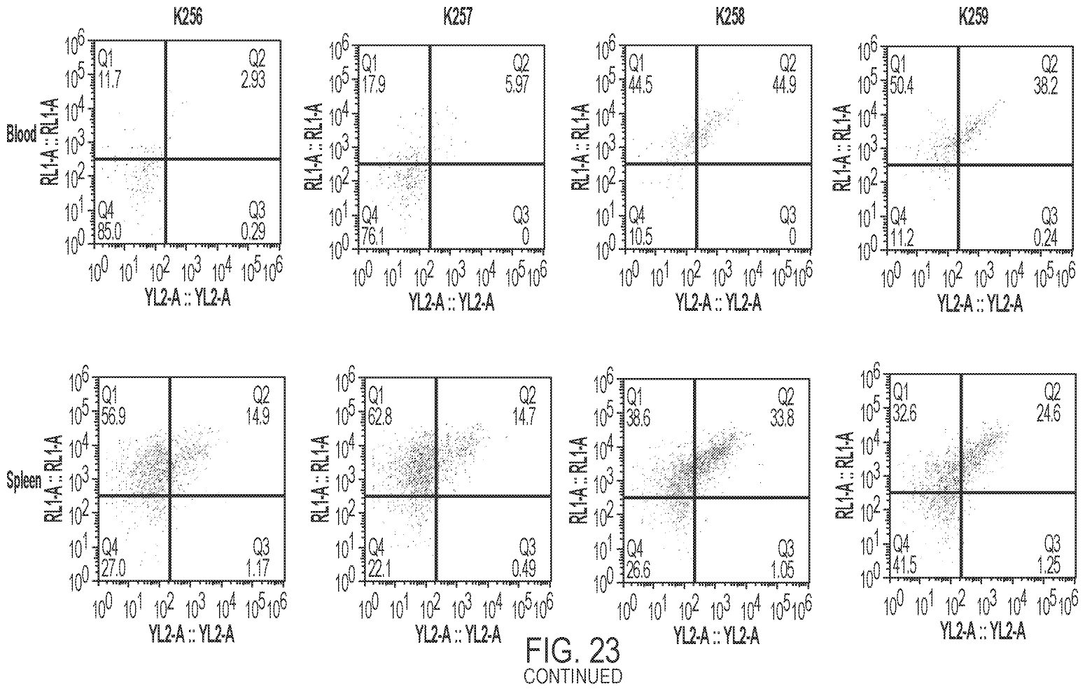

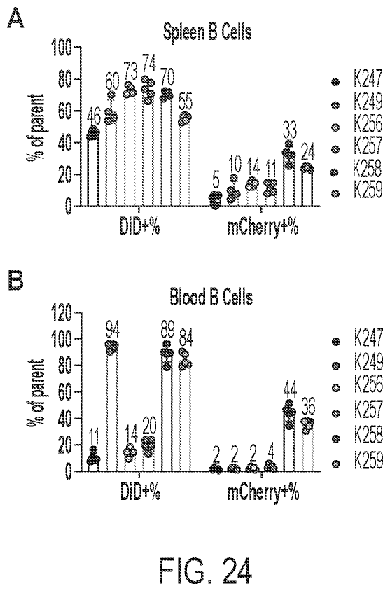

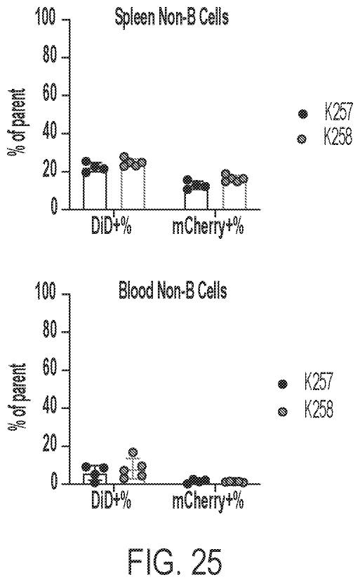

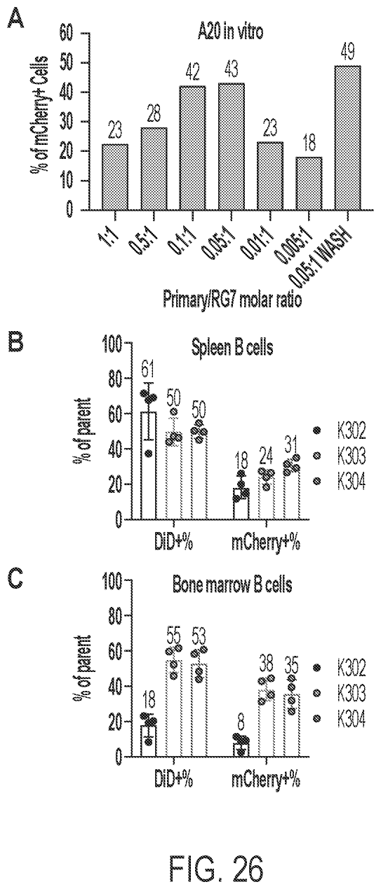

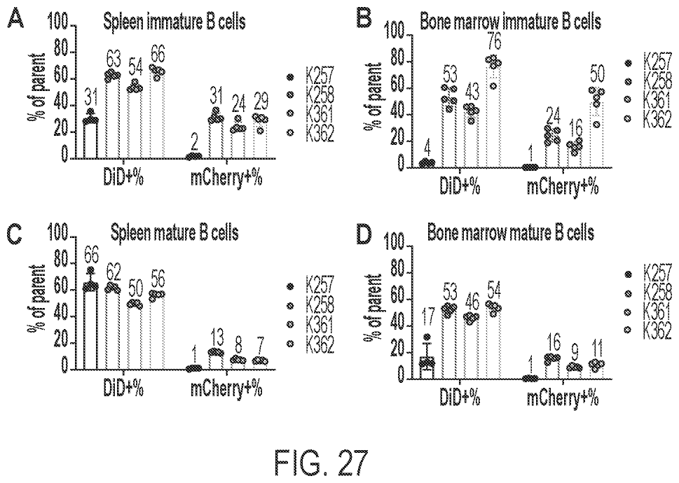

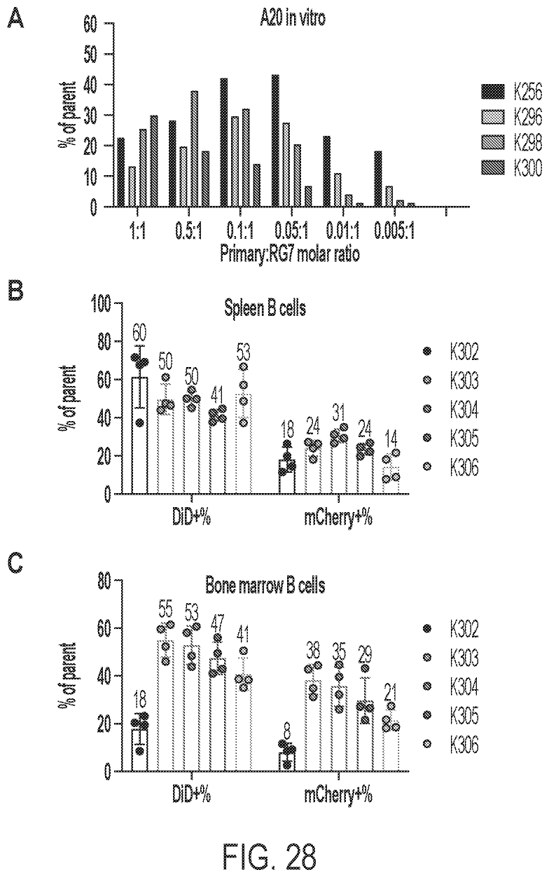

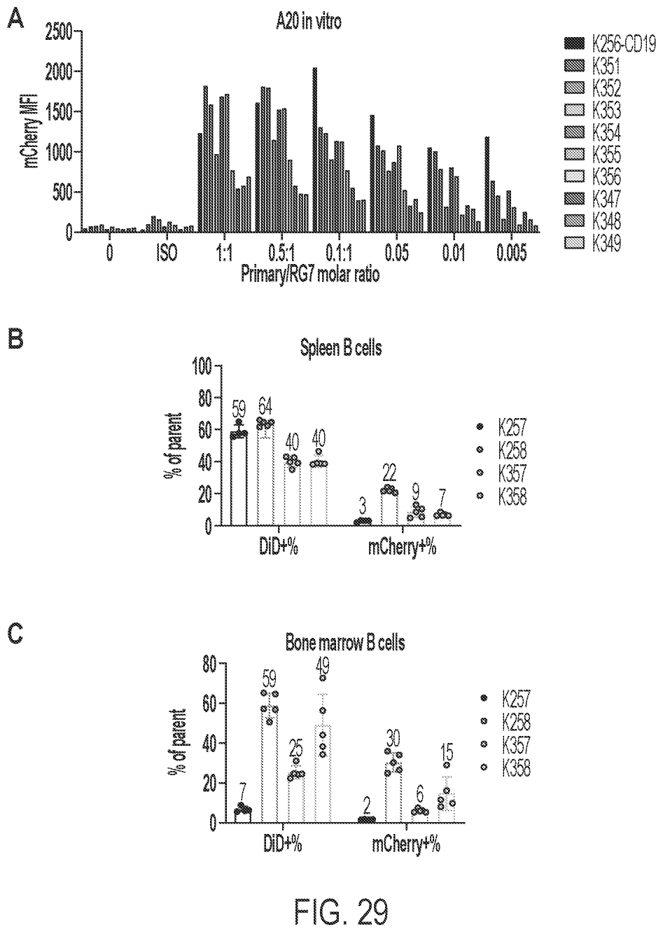

FIG. 3 shows ex vivo lung luminescence for the indicated LNPs 24 hrs post dosing.

FIG. 7 shows in vivo images of bioluminescence 18 hours after dosing of the indicated LNPs.

FIG. 8 shows ex vivo luminescence of the mice treated with indicated lipid compositions.

FIG. 9 shows ex vivo quantitation of bioluminescence for each LNP.

FIG. 10 shows ex vivo luminescence of the mice treated with indicated lipid compositions.

FIG. 11 shows ex vivo quantitation of bioluminescence for each LNP.

FIG. 12 shows in vivo images of the treatment groups.

FIG. 13A-13D shows quantification of luminescence of indicated regions and LNPs

FIG. 14 shows the distribution of the LNPs in whole body, liver, and tumor of indicated LNPs

FIG. 15 shows quantification of luminescence in tumors of indicated LNPs.

FIG. 16 shows quantification of luminescence in tumors of indicated LNPs.

FIG. 17 shows concentration of indicated LNPs in whole body and liver.

FIG. 18 shows an exemplary process for conjugating targeting entities (e.g., antibodies or fragments) to the surface of an LNP.

FIG. 19 shows fluorescence of conjugated antibodies at various indicated primary to secondary (RG7) ratios.

FIG. 20 shows transfection efficiency based on fluorescence of various functionalized LNPs.

FIG. 21 shows transfection efficiency based on fluorescence of various functionalized LNPs.

FIG. 22 shows transfection efficiency based on fluorescence of various functionalized LNPs.

FIG. 23 shows transfection efficiency based on fluorescence of various functionalized LNPs.

FIG. 24 shows transfection efficiency based on fluorescence of various functionalized LNPs.

FIG. 25 shows transfection efficiency of B cells and non-B cells based on fluorescence of various functionalized LNPs.

FIG. 26 shows targeting efficiency of indicated LNPs in B cells.

FIG. 27 shows targeting efficiency of indicated functionalized LNPs, adjusted for RG7/maleimide ratios, in various B cells.

FIGS. 28A-28C show targeting efficiency of indicated functionalized LNPs.

FIGS. 29A-29C show targeting efficiency of indicated functionalized LNPs.

### DEFINITIONS

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc., In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: As used herein, the term “agent”, may refer to a compound, molecule, or entity of any chemical class including, for example, a small molecule, polypeptide, nucleic acid, saccharide, lipid, metal, or a combination or complex thereof. In some embodiments, the term “agent” may refer to a compound, molecule, or entity that comprises a polymer. In some embodiments, the term may refer to a compound or entity that comprises one or more polymeric moieties. In some embodiments, the term “agent” may refer to a compound, molecule, or entity that is substantially free of a particular polymer or polymeric moiety. In some embodiments, the term may refer to a compound, molecule, or entity that lacks or is substantially free of any polymer or polymeric moiety.

Amelioration: as used herein, refers to the prevention, reduction or palliation of a state, or improvement of the state of a subject. Amelioration includes, but does not require complete recovery or complete prevention of a disease, disorder or condition (e.g., radiation injury).

Amino acid: in its broadest sense, as used herein, the term “amino acid” refers to a compound and/or substance that can be, is, or has been incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Antagonist: Those skilled in the art will appreciate that the term “antagonist”, as used herein, may be used to refer to an agent, condition, or event whose presence, level, degree, type, or form correlates with decreased level or activity of another agent (i.e., the inhibited agent, or target). In general, an antagonist may be or include an agent of any chemical class including, for example, small molecules, polypeptides, nucleic acids, carbohydrates, lipids, metals, and/or any other entity that shows the relevant inhibitory activity. In some embodiments, an antagonist may be direct (in which case it exerts its influence directly upon its target); in some embodiments, an antagonist may be indirect (in which case it exerts its influence by other than binding to its target; e.g., by interacting with a regulator of the target, so that level or activity of the target is altered).

Antibody: As used herein, the term “antibody” refers to an agent that specifically binds to a particular antigen. In some embodiments, the term encompasses a polypeptide or polypeptide complex that includes immunoglobulin structural elements sufficient to confer specific binding. For example, in some embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent in or comprises a polypeptide whose amino acid sequence includes structural elements recognized by those skilled in the art to correspond to CDRs1, 2, and 3 of an antibody variable domain; in some such embodiments, an antibody agent in or comprises a polypeptide or set of polypeptides whose amino acid sequence(s) together include structural elements recognized by those skilled in the art to correspond to both heavy chain and light chain variable region CDRs, e.g., heavy chain CDRs 1, 2, and/or 3 and light chain CDRs 1, 2, and/or 3. In some embodiments, an antibody agent is a polypeptide protein having a binding domain which is homologous or largely homologous to an immunoglobulin-binding domain. In some embodiments, an antibody agent may be or comprise a polyclonal antibody preparation. In some embodiments, an antibody agent may be or comprise a monoclonal antibody preparation. In some embodiments, an antibody agent may include one or more constant region sequences that are characteristic of a particular organism, such as a camel, human, mouse, primate, rabbit, rat; in many embodiments, an antibody agent may include one or more constant region sequences that are characteristic of a human. In some embodiments, an antibody agent may include one or more sequence elements that would be recognized by one skilled in the art as a humanized sequence, a primatized sequence, a chimeric sequence, etc. In some embodiments, an antibody agent may be a canonical antibody (e.g., may comprise two heavy chains and two light chains). In some embodiments, an antibody agent may be in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies;

masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINSR; Avimers®; DARTs; TCR-like antibodies;, Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.].

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of, susceptibility to, severity of, stage of, etc the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biologically active: as used herein, refers to an observable biological effect or result achieved by an agent or entity of interest. For example, in some embodiments, a specific binding interaction is a biological activity. In some embodiments, modulation (e.g., induction, enhancement, or inhibition) of a biological pathway or event is a biological activity. In some embodiments, presence or extent of a biological activity is assessed through detection of a direct or indirect product produced by a biological pathway or event of interest.

Cancer: The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Chemotherapeutic Agent: The term “chemotherapeutic agent”, has used herein has its art-understood meaning referring to one or more pro-apoptotic, cytostatic and/or cytotoxic agents, for example specifically including agents utilized and/or recommended for use in treating one or more diseases, disorders or conditions associated with undesirable cell proliferation. In many embodiments, chemotherapeutic agents are useful in the treatment of cancer. In some embodiments, a chemotherapeutic agent may be or comprise one or more alkylating agents, one or more anthracyclines, one or more cytoskeletal disruptors (e.g. microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some particular embodiments, a chemotherapeutic agent may be or comprise one or more of Actinomycin, All-trans retinoic acid, an Auiristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g. DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, and combinations thereof. In some embodiments, a chemotherapeutic agent may be utilized in the context of an antibody-drug conjugate. In some embodiments, a chemotherapeutic agent is one found in an antibody-drug conjugate selected from the group consisting of: hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Designed: As used herein, the term “designed” refers to an agent (i) whose structure is or was selected by the hand of man; (ii) that is produced by a process requiring the hand of man; and/or (iii) that is distinct from natural substances and other known agents.

Encapsulated: The term “encapsulated” is used herein to refer to substances that are completely surrounded by another material.

Engineered: In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature. For example, in some embodiments of the present invention, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

Excipient: as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc); (3) translation of an RNA into a polypeptide or protein; and/or (4) post-translational modification of a polypeptide or protein.

Human: In some embodiments, a human is an embryo, a fetus, an infant, a child, a teenager, an adult, or a senior citizen.

“Improved,” “increased” or “reduced”: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

In vitro: The term “in vitro” as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and a non-human animal. In the context of cell-based systems, the term may be used to refer to events that occur within a living cell (as opposed to, for example, in vitro systems)

Nanoparticle: As used herein, the term “nanoparticle” refers to a discrete entity of small size, e.g., typically having a longest dimension that is shorter than about 1000 nanometers (nm) and often is shorter than 500 nm, or even 100 nm or less. In many embodiments, a nanoparticle may be characterized by a longest dimension between about 1 nm and about 100 nm, or between about 50 nm to 200 nm, or between about 1 μm and about 500 nm, or between about 1 nm and 1000 nm. In many embodiments, a population of nanoparticles is characterized by an average size (e.g., longest dimension) that is below about 1000 nm, about 750 nm, about 500 nm, about 200 nm, about 100 nm, about 50 nm, about 40 nm, about 30 nm, about 20 nm, or about 10 nm and often above about 1 nm. In many embodiments, a nanoparticle may be substantially spherical (e.g., so that its longest dimension may be its diameter). In some embodiments, a nanoparticle has a diameter of less than 100 nm as defined by the National Institutes of Health. In some embodiments, nanoparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Payload: In general, the term “payload”, as used herein, refers to an agent that may be delivered or transported by association with another entity. In some embodiments, such association may be or include a covalent linkage; in some embodiments such association may be or include non-covalent interaction(s). In some embodiments, association may be direct; in some embodiments, association may be indirect. The term “payload” is not limited to a particular chemical identity or type; for example, in some embodiments, a payload may be or comprise, for example, an entity of any chemical class including, for example, a lipid, a metal, a nucleic acid, a polypeptide, a saccharide (e.g., a polysaccharide), small molecule, or a combination or complex thereof. In some embodiments, a payload may be or comprise a biological modifier, a detectable agent (e.g., a dye, a fluorophore, a radiolabel, etc.), a detecting agent, a nutrient, a therapeutic agent, etc., or a combination thereof. In some embodiments, a payload may be or comprise a cell or organism, or a fraction, extract, or component thereof. In some embodiments, a payload may be or comprise a natural product in that it is found in and/or is obtained from nature; alternatively or additionally, in some embodiments, the term may be used to refer to one or more entities that is man-made in that it is designed, engineered, and/or produced through action of the hand of man and/or is not found in nature. In some embodiments, an payload may be or comprise an agent in isolated or pure form; in some embodiments, such agent may be in crude form.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Suffering from: An individual who is “suffering from” a disease, disorder, and/or condition displays one or more symptoms of a disease, disorder, and/or condition and/or has been diagnosed with the disease, disorder, or condition.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. In some embodiments, treatment may be phrophylactic; for example may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition and/or for delaying onset or decreasing rate of development or worsening of one or more features of a disease, disorder and/or condition.

### DETAILED DESCRIPTION OF CERTAIN EMBODIMENTS

### Lipid Nanoparticles

In some embodiments, the present disclosure provides lipid nanoparticles (LNP). The present disclosure provides, at least in part, the discovery that certain lipid components of a lipid nanoparticle enhance association of a LNP with particular tissues. In some embodiments, the present disclosure provides lipid nanoparticles which exhibit increased delivery of a payload to specific tissues relative to other tissues. In some embodiments, the present disclosure provides lipid nanoparticles which exhibit tropism for specific tissues relative to other tissues.

In some embodiments, the present disclosure provides LNPs comprising at least one cationic lipid. In some embodiments, the present disclosure provides LNPs comprising at least one cationic ionizable lipid. In some embodiments, the term “cationic ionizable lipid” refers to lipid and lipid-like molecules with nitrogen atoms that can acquire charge (pKa). In some embodiments, a cationic ionizable lipid for use in accordance with the present disclosure has a pKa of 5, 6, 7, 8, 9, 10, 11 at physiological pH. In some embodiments a cationic ionizable lipid comprises one or more groups which is protonated at physiological pH but deprotonates and has no charge at a pH above 5, 6, 7, 8, 9, 10, 11, or 12. In some embodiments a cationic ionizable lipid comprises one or more groups which are protonated and have a charge at a pH above 5, 6, 7, 8, 9, 10, 11, or 12. In some embodiments, the ionizable cationic group may contain one or more protonatable amines which are able to form a cationic group at physiological pH.

One insight provided by the present disclosure is that use of a cationic ionizable lipid with a pKa within a particular range as described herein may be particularly useful for delivery of nucleic acids, for example in LNP preparations as described herein. Specifically, in some embodiments, a cationic ionizable lipid has a high pKA. In some embodiments a high pKa is a pKa greater than 7. In some embodiments a high pKa is a pKa greater than 7.4. In some embodiments, a cationic ionizable lipid for use in accordance with the present disclosure has a pKa of between 7 and 8, 7.5 and 8.5, 8 and 9, 8.5 and 9.5, 9 and 10. In some embodiments, a cationic ionizable lipid for use in accordance with the present disclosure has a pKa of between 7.2 and 8.2, 7.4 and 8.4, 7.6 and 8.6, 7.8 and 8.8, 8.0 to 9.0, 8.2 to 9.2, 8.4 to 9.4, 8.6 to 9.6, 8.8 to 9.8, 9.0 to 10.0. In some embodiments, a useful cationic ionizable lipid has a pKa of 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. This insight is particularly surprising in light of the understanding in the field that pKas >7 are not optimum for LNP payload delivery. See Jayaraman et al., Angew. Chem. Int. Ed. 2012, 51, 8529-8533. Indeed, Jayaraman et al states that LNP potency rapidly decreases if the pKa is outside of the range of 6.2 to 6.5. Further, with respect to intramuscular administration of LNP Hassett et al., Molecular Therapy: Nucleic Acids Vol. 15 Apr. 2019 staes that a lipid pKa range of 6.6-6.9 is optimal.

In some embodiments, a cationic ionizable lipid with a high pKA (e.g., >7) is disclosed or described in WO2020219876; US20210162053; US20160317676; and WO2021113365 each of which is incorporated herein in their entirety. In some embodiments, a cationic ionizable lipid with a high pKA (e.g., >7 is one selected from those listed in Table 1.

| | |
|---|---|
| 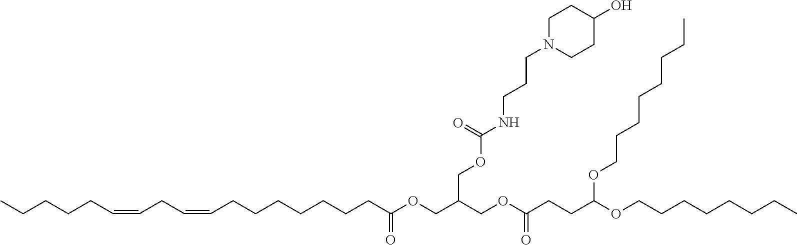 | 3-((4,4- bis(octyloxy) butanoyl) oxy)-2- ((((3-(4- hydroxy- piperidin-1- yl)propyl) carbamoyl) oxy)methyl) propyl (9Z,12Z)- octadeca-9,12- dienoate |
| (LIP021; also referred to as C1 herein)) | |
| 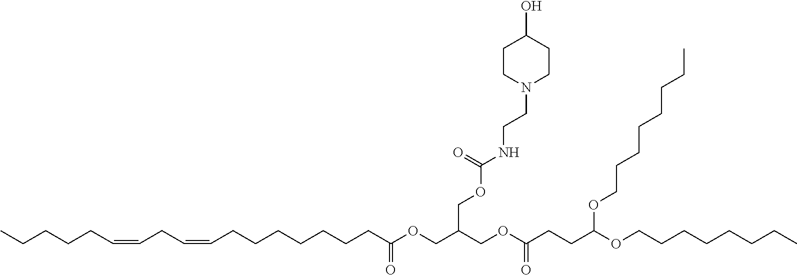 | 3-((4,4- bis(octyloxy) butanoyl) oxy)-2- ((((2-(4- hydroxy- piperidin-1- yl)ethyl) carbamoyl) oxy)methyl) propyl (9Z,12Z)- octadeca- 9,12- dienoate |
| (LIP057, also referred to as C2 herein) | |
| 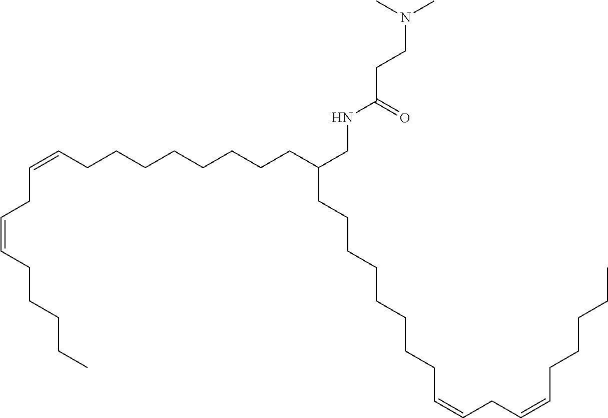 | 3-(dimethyl- amino)- N-((11Z,14Z)- 2-((9Z,12Z)- octadeca-9,12- dienyl)icosa- 11,14- dienyl) propanamide |
| (LIP058, ALYN-201) | |
| 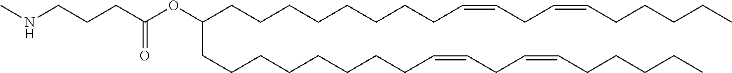 | DLin-M- C3-MA |
| (LIP019, DLin-M-C3-MA, also referred to as A1 | |
| herein) | |
| 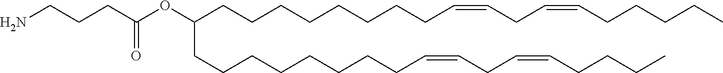 | DLin-M- C3-A |
| (LIP059, DLin-M-C3-A) | |
| 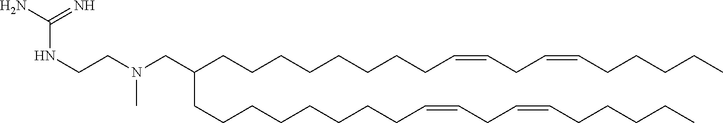 | Same as D1 |
| (LIP022, ALYN-139, also referred to as D1 herein) | |
| 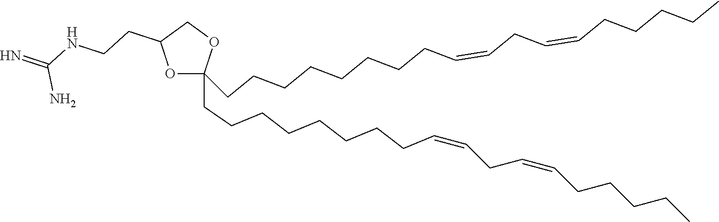 | |
| (LIP060, AYLN-232) | |
| 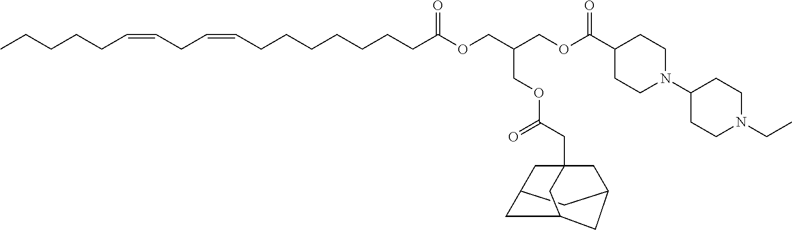 | |
| (LIP020, also referred to as B1 herein) | |
| 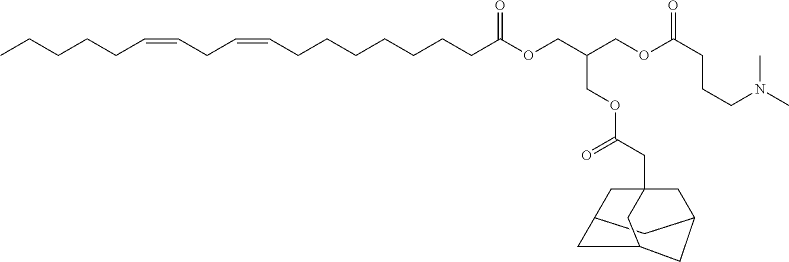 | |
| (LIP061, also referred to as B2 herein) | |
| 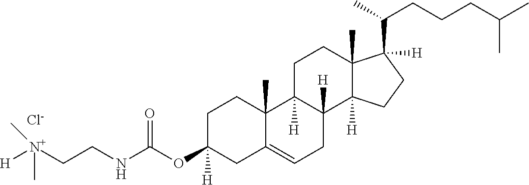 | DC- Cholesterol |
| (LIP005, DC-Cholesterol) | |
| 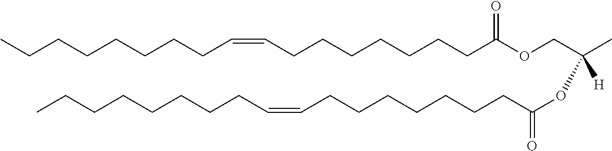 | DOTAP |
| (LIP027, DOTAP) | |
| 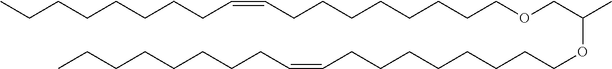 | DOTMA |
| (LIP028, DOTMA) | |

Among other things, the present disclosure demonstrates that use of a high pKa cationically ionizable lipid in LNPs as described herein may be particularly useful to achieve delivery of nucleic acids to particular tissues. In some embodiments, LNPs as described herein may be particularly useful to achieve delivery of nucleic acids to the lung. In some embodiments, LNPs as described herein may be particularly useful to achieve delivery of nucleic acids to tissues other than the liver (i.e., extrahepatic delivery). In some embodiments, LNPs as described herein may be particularly useful to achieve delivery of nucleic acids to tumors. In some embodiments, LNPs as described herein may be particularly useful to achieve delivery of nucleic acids to B cells.

In some embodiments the present disclosure provides LNPs comprising a at least first cationic ionizable lipid and a second cationic ionizable lipid. In some such embodiments, more than one (e.g., each) of such at least first and second cationic ionizable lipids has a high pKa (e.g., greater than 7, e.g., greater than 7.4) as described herein. Alternatively, in some such embodiments, only one (i.e., a first) cationic ionizable lipid has such a high pKa (e.g., greater than 7, e.g., greater than 7.4) as described herein. In some embodiments, a second cationic ionizable lipid is a non-high-pKa lipid, e.g., in that it has a pKa below 7, e.g., about 6.8, 6.6, 6.4, 6.2, 6.0, 5.8, 5.6, 5.4, 5.2, 5.0 or lower. In some embodiments, a non-high-pKa lipid (e.g., a second lipid) has a pKa of about 6.4 or lower. In some embodiments, a non-high-pKa lipid (e.g., a second lipid) has a pKa of about 6.4. In some embodiments, a non-high-pKa lipid (e.g., a second lipid) has a neutral pKa.

In some embodiments, a cationic ionizable lipid with a non-high pKa (e.g., <7) is disclosed or described in Finn et al., 2018 Cell Reports 22, 2227-2235; Jayaraman et al., Angew. Chem. Int. Ed. 2012, 51, 8529-8533; Hassett et al., Molecular Therapy: Nucleic Acids Vol. 15 Apr. 2019; WO2015074085; WO2020118041; WO2020072605; WO2020252589; WO2021055849; WO2018232120; WO2021030701; WO2020146805; WO2019036000; WO2018200943; and WO2018191657 each of which is incorporated herein in their entirety. In some embodiments, a cationic ionizable lipid with a non-high pKA (e.g., <7) is one selected from those listed in Table 4

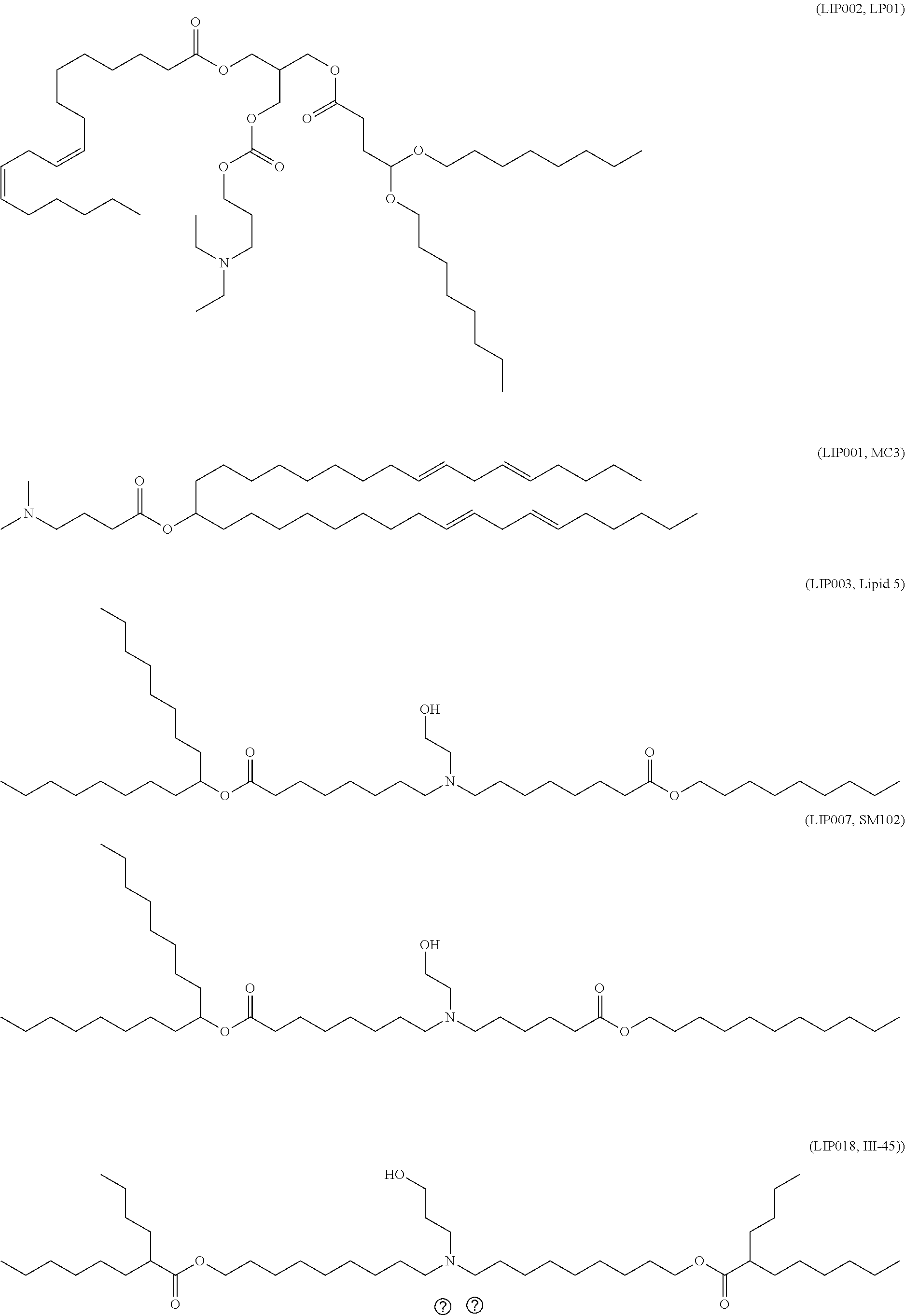

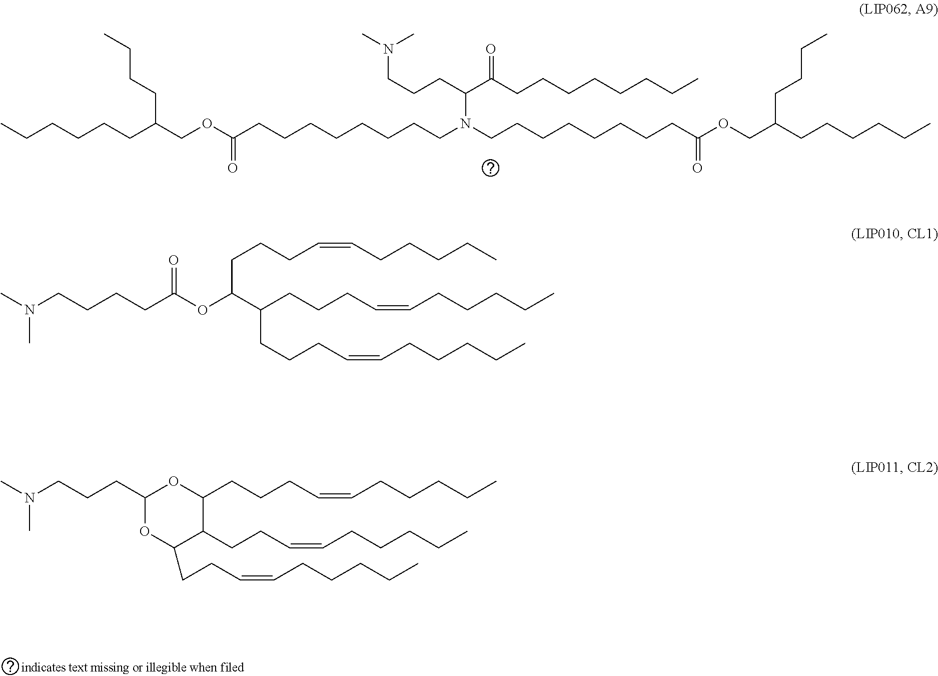

In some embodiments, an LNP comprises about 0-80 mol % of high pKa cationic ionizable lipid as described herein; in some embodiments, an LNP comprises two or more high pKa cationic ionizable lipids that, together make up such 0-80 mol % of the LNP. In some embodiments, an LNP comprises about 20-80 mol % of high pKa cationic ionizable lipid. In some embodiments, an LNP comprises about 0, 5, 10, 15, 20, 25, 30, 35, 40, 45, 50, 55, 60, 65, 7-, 75, 80 mol % of high pKa cationic ionizable lipid.

In some embodiments, an LNP comprises about 0-60 mol % of cationic ionizable lipid that is not high pKa (e.g., that is characterized by a pKa below about 7, such as a pKa of about 6.4 or a neutral pKa); in some embodiments, an LNP comprises two or more non-high pKa cationic ionizable lipids that, together, make up such 0-60 mol % of the LNP. In some embodiments, an LNP comprises about 0, 5, 10, 15, 20, 25, 30, 35, 40, 45, 50, 55, 60 mol % of a non-high-pKa cationic ionizable lipid.

In some embodiments, the present disclosure provides LNPs comprising at least one sterol. In some embodiments, an LNP comprises about 0-40 mol % of a sterol. In some embodiments, an LNP comprises about 0, 5, 10, 15, 20, 25, 30, 35, 40 mol % of a sterol.

In some embodiments, a sterol is cholesterol, or a variant or derivative thereof. In some embodiments, a cholesterol is modified, for example oxidized. Unmodified cholesterol can be acted upon by enzymes to form variants that are side-chain or ring oxidized. In some embodiments, a cholesterol can be oxidized on the beta-ring structure or on the hydrocarbon tail structure. Exemplary cholesterols that are considered for use in the disclosed LNPs include but are not limited to 25-hydroxycholesterol (25-OH), 20α-hydroxycholesterol (20α-OH), 27-hydroxycholesterol, 6-keto-5α-hydroxycholesterol, 7-ketocholesterol, 7β-hydroxycholesterol, 7α-hydroxycholesterol, 7β-25-dihydroxycholesterol,beta-sitosterol, stigmasterol, brassicasterol, campesterol, or combinations thereof. In some embodiments, side-chain oxidized cholesterol can enhance cargo delivery relative to other cholesterol variants. In some embodiments, a cholesterol is an unmodified cholesterol.

In some embodiments, the present disclosure provides LNPs comprising at least one helper lipid. In some embodiments a helper lipid is a phospholipid. In some embodiments, an LNP comprises about 0-20 mol % of a helper lipid. In some embodiments, an LNP comprises about 0, 5, 10, 15, 20 mol % of a helper lipid.

Exemplary phospholipids include but are not limited to 1,2-distearoyl-snglycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycerophosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycerophosphocholine (DUPC), 1-palmitoyl-2-oleoyl-sn-glycero-3-phosphocholine (POPC), 1,2-di-0-octadecenyl-sn-glycero-3-phosphocholine (18:0 Diether PC), 1-oleoyl-2-cholesterylhemisuccinoy 1-sn-glycero-3-phosphocholine (OChemsPC), 1-hexadecyl snglycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidy ethanolamine (SOPE), 1-stearoyl-2 oleoylphosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), or combinations thereof. In some embodiments, a phospholipid is DSPC. In some embodiments, a phospholipid is DMPC.

In some embodiments, a phospholipid comprises 1,2-dioleoyl-sn-glycero-3-phosphoethanolamine-N-(succinyl) (succinyl PE), 1,2-distearoyl-sn-glycero-3-phosphocholine (DSPC), cholesterol, 1,2-distearoyl-sn-glycero-3-phosphoethanolamine (DSPE), 1,2-dipalmitoyl-sn-glycero-3-phosphoethanolamine-N-(succinyl) (succinyl-DPPE), 1,2-dioleoyl-sn-glycero-3-phosphoethanolamine (DOPE), 1,2-dimyristoyl-sn-glycero-3-phosphocholine (DMPC), 1,2-dipalmitoyl-sn-glycero-3-phosphocholine (DPPC), or a combination thereof.

In some embodiments, the present disclosure provides LNPs comprising at least one PEGylated lipid. In some embodiments, an LNP comprises about 0-25 mol % of a PEGylated lipid. In some embodiments, an LNP comprises about 0, 5, 10, 15, 20, 25 mol % of a PEGylated lipid.

In some embodiments, inclusion of a PEGylated lipid can be used to enhance lipid nanoparticle colloidal stability in vitro and circulation time in vivo. In some embodiments, PEGylation is reversible in that the PEG moiety is gradually released in blood circulation. Exemplary PEGylated-lipids include but are not limited to PEG conjugated to saturated or unsaturated alkyl chains having a length of C6-C20, PEG-modified-28-phosphatidylethanolamines, PEG-modified phosphatidic acids, PEG-modified ceramides (PEG-CER), PEG-modified dialkylamines, PEG-modified diacylglycerols (PEG-DAG), PEG-modified dialkylglycerols, and mixtures thereof. For example, a PEGylated-lipid may be PEG-c-DOMG, PEG-DMG, PEG-DLPE, PEG-DMPE, PEG-DPPE, PEG-DSG or a PEGDSPE lipid.

In some embodiments, the present disclosure provides LNPs comprising at least one PEG-ligand. In some embodiments, an LNP comprises about 0-2 mol % of a PEG-ligand. In some embodiments, an LNP comprises about 0, 0.01, 0.05, 0.1, 0.5, 0.75, 1, 1.5, 1.75, 2 mol % of a PEG-ligand. In some embodiments, a ligand (e.g., as included in a PEG-ligand) is or comprises: antibodies targeting cell surface proteins, hyaluronic acid, small molecules, peptides, and/or peptides that target integrins.

### Functionalized LNPs

The present disclosure provides LNPs directed to particular tissues. In some embodiments LNPs of the present disclosure are directed to particular tissues by functionalization of the LNPs.

In some embodiments, LNPs of the present disclosure are functionalized by post-insertion of PEG-derivatized targeting ligands. In some embodiments, an LNP comprises about 0-10 mol % of a PEG-ligand. In some embodiments, an LNP comprises about 0, 0.01, 0.05, 0.1, 0.5, 0.75, 1, 1.5, 1.75, 2, 3, 4, 5, 6, 7, 8, 9, 10 mol % of a PEG-ligand. In some embodiments, a ligand (e.g., as included in a PEG-ligand) is or comprises anisamide, folate, hyaluronic acid 10K, hyaluronic acid 100K, transferrin, or other small molecules and peptides.

In some embodiments, LNPs of the present disclosure are functionalized by conjugation of a targeting entity to the surface of a LNP described herein. In in some embodiments LNPs of the present disclosure are functionalized by conjugation of a targeting entity to PEG-derivatized lipids. Exemplary PEG-derivatized lipids include but are not limited to PEG-DMG, PEG-DSPE, PEG-DSG, PEG-DMG, PEG-maleimide-PEG-DSPE, maleimide-PEG-DMG, Azide-PEG-DSPE, DBCO-PEG-DSPE, NH2-PEG-DSPE, COOH-PEG-DSPE, or combinations thereof. PEG molecular weights include but are not limited to 2000, 3000, 3500, 4000, and 5000. In some embodiments, an LNP comprises about 0, 0.01, 0.05, 0.1, 0.5, 0.75, 1, 1.5, 1.75, 2, 3 mol % of each or a combination of PEG-derivatized lipids.

In some embodiments, a targeting entity is an antibody (e.g., a monoclonal antibody) or antibody fragment (e.g., a binding fragment). In some embodiments, a targeting entity comprisies a primary antibody and a secondary antibody. In some embodiments a secondary antibody or fragment binds an Fc region. In some embodiments, a secondary antibody or fragment is or comprises an anti-human Fc, anti-mouse Fc, anti-rabbit Fc, or anti-hamster Fc antibody or antibody fragment. In some embodiments, a secondary antibody or fragment is or comprises, RG7/1.30 for Fc domain of Rat IgG2a, RG7/11.1 for Fc domain of Rat IgG2b. In some embodiments a prirmary antibody or fragment is an antibody or fragment that binds or targets a specific antigen of the desired cell type or tissue. In some embodiments, a primary antibody or fragment binds or targets a B cell surface receptor. In some embodiments, a primary antibody or fragment binds or targets CD19, CD20, CD23, CD38, or CD138.

In some embodiments, the molar ratios of antibody or antibody fragment to PEG-derivatized lipids are adjusted to achieve the maximal targeting effect. In some embodiments, the molar ratios of antibody or antibody fragment to PEG-derivatized lipids include but not limit to 0.001, 0.005, 0.01, 0.05, 0.1, 0.2, 0.3, 0.4, 0.5, 0.6, 0.7, 0.8, 0.9 and 1.

In some embodiments, antibody or antibody fragment are modified by DTT, TCEP, SATA ((N-succinimidyl S-acetylthioacetate), SATP (N-succinimidyl-S-acetylthiopropionate), NHS-PEG₄-DBCO, NHS-PEG₄-Azide, or NHS-PEG₄-ester to expose functional groups such as sulfhydryl, DBCO, and Azide groups. In some embodiments, an antibody or antibody fragment contains 0, 1, 2, 3, 4, 5, 6, 7, 8, 9 and 10 functional groups.

### Payload

In some embodiments, LNPs of the present disclosure comprise a payload. In some embodiments a payload is an agent. In some embodiments a payload is a therapeutic or detection agent.

In some embodiments, a therapeutic agent is a nucleic acid. Exemplary nucleic acids may be or include deoxyribonucleic acid (DNA), ribonucleic acid (RNA) RNA, analogs, and/or combinations thereof. For example, in some embodiments, a nucleic acid may be or comprise single-stranded RNA, single-stranded DNA, double-stranded RNA, double stranded DNA, triple-stranded DNA, siRNA, shRNA, sgRNA, mRNA, miRNA, and/or antisense DNA. In some embodiments, a nucleic acid may include one or more non-natural residues as is known in the art.

In some embodiments, a nucleic acid (e.g., an RNA) may be sequence engineered, for example to remove immunogenic sequence motifs. In some embodiments, a nucleic acid is sequence engineered to remove TLR7 or TLR8 stimulation motifs. In some embodiments, a nucleic acid is sequence engineered to remove motifs selected from the group consisting of KNUNDK motifs, UCW motifs, UNU motifs, UWN motifs, USU motifs, KWUNDK motifs, KNUWDK motifs, UNUNDK motifs, KNUNUK motifs, and combinations thereof. In some embodiments, a nucleic acid is sequence engineered as described in WO2020/033720 the entirety of which is incorporated herein by reference.

In some embodiments, a nucleic acid is preferentially or selectively expressed (e.g., transcribed and/or translated) in a tumor; in some such embodiments, a nucleic acid is an RNA that is preferentially or selectively translated in a tumor. In some embodiments, a nucleic acid of the present disclosure comprises a translatable sequence comprising an oncoselective translation sequence element (e.g., an oncoselective readthrough motif) aa described in WO2020/257655 the entirety of which is incorporated herein by reference.

### Uses

In some embodiments, LNPs of the present disclosure target or attach to specific tissues. In some embodiments, the present disclosure recognizes that inclusion of cationic ionizable lipids in an LNP results in more effective targeting of LNPs and thus delivery of a payload to specific tissues or cells. In some embodiments, LNPs as described herein preferentially target tissues other than tissues (i.e., extrahepatic). In some embodiments, LNPs as described herein preferentially target lung tissue or lung cells relative to other tissues. In some embodiments, LNPs as described herein preferentially target lung tissue and/or lung cells, e.g., relative to liver tissue or cells. In some embodiments, LNPs as described herein preferentially target tumor tissue or tumor cells (e.g., cancer cells) relative to other tissues. In some embodiments, LNPs as described herein preferentially target tumor tissue and/or tumor cells, e.g., relative to liver tissue or cells.

In some embodiments, provided LNPs achieve extrahepatic delivery at a level that is significantly higher than that observed with an appropriate reference LNP. In some embodiments, provided LNPs achieve tumor delivery at a level that is significantly higher than that observed with an appropriate reference LNP. In some embodiments, provided LNPs achieve lung delivery at a level that is significantly higher than that observed with an appropriate reference LNP.

In some embodiments, provided LNPs achieve preferential extrahepatic, tumor, spleen, and/or lung delivery relative to liver delivery to a degree greater than that observed with an appropriate reference LNP. For example, the present Examples document that, in some embodiments, an RNA delivered with a provided LNP is preferentially expressed in whole body, tumor, spleen, and/or lung relative to liver at ratios that may be 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000 or more greater than those observed with a reference LNP (e.g., LNP 1 in Example 1). In some embodiments, whole body: liver; tumor: liver, spleen: liver, and/or lung: liver expression ratio is 1, 2, 3, or 4 orders of magnitude greater for a provided LNP than for a relevant reference LNP.

In some embodiments, provided LNPs achieve whole body, tumor, and/or lung delivery to a level that is as least 10%, 15%, 20%, 25%, 30%, 35%, 40%, 45%, 50%, 55%, 60%, 65%, 70%, 75%, 80%, 85%, 90%, 95%, or more of its level of liver delivery. In some embodiments, provided LNPs achieve whole body, tumor, and/or lung delivery that is reasonably comparable to its liver delivery. In some embodiments, provided LNPs achieve preferential whole body, tumor, and/or lung delivery relative to liver delivery. In some embodiments, provided LNPs achieve whole body, tumor, and/or lung delivery at a level that is at least 2, 3, 4, 5, 6, 7, 8, 9, 10, 15, 20, 25, 30, 35, 40, 45, 50, 55, 60, 65, 70, 75, 80, 85, 90, 95 or 100 fold or more that of its level of liver delivery.

In some embodiments, provided LNPs achieve both material levels of whole body, tumor, and/or lung expression and significant preference for delivery to whole body, tumor, and/or lung e.g., relative to liver (i.e., extrahepatic). In some embodiments, LNPs as described herein delivering nucleic acids to extrahepatic tissues can be selected from one of the following compositions:

| Formulation | Composition |
|---|---|
| K029 | 50 mol % LIP002; 27.5% cholesterol; 22 |
| | mol % DSPC; 0.5 mol % PEG-DMG. |
| K030 | 50 mol % LIP003; 27.5% cholesterol; 22 |
| | mol % DSPC; 0.5 mol % PEG-DMG |
| K108 | 50 mol % LIP003; 27.5% cholesterol; 22 |
| | mol % DSPC; 0.5 mol % PEG-DSPE |
| K111 | 50 mol % LIP003; 25% cholesterol; 22 |
| | mol % DSPC; 3 mol % FA-PEG-DSPE |
| K051 | 46.8 mol % LIP003; 36.8% cholesterol; 15.9 |
| | mol % DSPC; 0.5 mol % PEG-DMG |
| K052 | 46.8 mol % LIP003; 24.9% cholesterol; 27.9 |
| | mol % DSPC; 0.5 mol % PEG-DMG |
| K053 | 34.8 mol % LIP003; 36.8% cholesterol; 27.9 |
| | mol % DSPC; 0.5 mol % PEG-DMG |
| K054 | 42.8 mol % LIP003; 32.8% cholesterol; 23.9 |
| | mol % DSPC; 0.5 mol % PEG-DMG |

In some embodiments, LNPs as described herein can be used to treat a subject suffering from a disease. In some embodiments, LNPs as described herein can be used to treat diseases of the lung. In some embodiments, LNPs as described herein can be used to deliver therapeutic agents for the treatment of diseases of the lung. In some embodiments, LNPs as described herein can be used to deliver nucleic acids for the treatment of diseases of the lung. In some embodiments, LNPs as described herein delivering nucleic acids to the lung can be selected from one of the following compositions:

| Formulation | Composition |
|---|---|
| K056 | 50 mol % DOTAP, 25 mol % LIP001; |
| | 18.75% cholesterol; 5 mol % DSPC; 0.75 |
| | mol % PEG-DMG, 0.5% PEG-ligand |
| K057 | 50 mol % DOTAP, 25 mol % LIP001; |
| | 19.15% cholesterol; 5 mol % DSPC; 0.75 |
| | mol % PEG-DMG, 0.1% PEG-ligand |
| K058 | 50 mol % DOTAP, 25 mol % LIP001; |
| | 19.2% cholesterol; 5 mol % DSPC; 0.75 |
| | mol % PEG-DMG, 0.05% PEG-ligand |
| K099: | 50 mol % DOTAP, 25 mol % LIP003; |
| | 18.5% cholesterol; 5 mol % DSPC; 0.5 |
| | mol % PEG-DMG, 1% PEG-ligand |
| K100 | 48.2 mol % DOTAP, 24.1 mol % LIP003; |
| | 17.8% cholesterol; 4.8 mol % DSPC; 4 |
| | mol % PEG-DMG, 1% PEG-ligand |
| K101 | 45.7 mol % DOTAP, 22.8 mol % LIP003; |
| | 16.9% cholesterol; 4.6 mol % DSPC; 9 |
| | mol % PEG-DMG, 1% PEG-ligand |
| K102 | 43.1 mol % DOTAP, 21.6 mol % LIP003; |
| | 16% cholesterol; 4.3 mol % DSPC; 14 |
| | mol % PEG-DMG, 1% PEG-ligand |
| K261 | 50 mol % LIP019; 25 mol % LIP003; |
| | 19.25% cholesterol; 5 mol % DSPC; 0.75 |
| | mol % PEG-DMG. |
| K263 | 50 mol % LIP021; 25 mol % LIP003; |
| | 19.25% cholesterol; 5 mol % DSPC; 0.75 |
| | mol % PEG-DMG |
| K269 | 50 mol % LIP021; 25 mol % LIP001; |
| | 19.25% cholesterol; 5 mol % DSPC; 0.75 |
| | mol % PEG-DMG |

In some embodiments, LNPs as described herein can be used to treat cancer. In some embodiments, LNPs as described herein can be used to deliver therapeutic agents for the treatment of cancer. In some embodiments, LNPs as described herein can be used to deliver nucleic acids for the treatment of cancer. In some embodiments, LNPs as described herein can be used to deliver therapeutic agents to tumors. In some embodiments, LNPs as described herein can be used to deliver nucleic acids to tumors.

In some embodiments, LNPs as described herein can be used to treat diseases of B cells. In some embodiments, LNPs as described herein can be used to deliver therapeutic agents to B cells. In some embodiments, LNPs as described herein can be used to deliver nucleic acids to B cells. In some embodiments, LNPs as described herein delivering nucleic acids to B cells can be selected from one of the following compositions:

| Formulation | Composition |
|---|---|
| K258 (DTT) | 50 mol % LIP003; 27.5% cholesterol; 22 |
| | mol % DSPC; 0.5 mol % Maleimide-PEG- |
| | DSPE, RG7/Maleimide molar ratio = 0.32/1, |
| | CD19 antibody/RG7 weight ratio = 0.1/1 |
| K259 (DTT) | 50 mol % LIP003; 27.5% cholesterol; 22 |
| | mol % DSPC; 0.5 mol % Maleimide-PEG- |
| | DSPE, RG7/Maleimide molar ratio = 0.32/1, |
| | CD19 antibody/RG7 weight ratio = 1/1 |
| K305 (DTT) | 50 mol % LIP003; 27.5% cholesterol; 22 |
| | mol % DSPC; 0.5 mol % Maleimide-PEG- |
| | DSPE, RG7/Maleimide molar ratio = 0.32/1, |
| | CD19 antibody/RG7 molar ratio = 0.5/1 |
| K306 (DTT) | 50 mol % LIP003; 27.5% cholesterol; 22 |
| | mol % DSPC; 0.5 mol % Maleimide-PEG- |
| | DSPE, RG7/Maleimide molar ratio = 0.32/1, |
| | CD19 antibody/RG7 molar ratio = 0.5/1 |
| K361 (DTT) | 50 mol % LIP003; 27.5% cholesterol; 22 |
| | mol % DSPC; 0.5 mol % Maleimide-PEG- |
| | DSPE, RG7/Maleimide molar ratio = 0.16/1, |
| | CD19 antibody/RG7 molar ratio = 0.1/1 |
| K362 (DTT) | 50 mol % LIP003; 27.5% cholesterol; 22 |
| | mol % DSPC; 0.5 mol % Maleimide-PEG- |
| | DSPE, RG7/Maleimide molar ratio = 0.06/1, |
| | CD19 antibody/RG7 molar ratio = 0.5/1 |
| K305 (SATA) | 50 mol % LIP003; 27.5% cholesterol; 22 |
| | mol % DSPC; 0.5 mol % Maleimide-PEG- |
| | DSPE, RG7/Maleimide molar ratio = 0.02/1, |
| | CD19 antibody/RG7 molar ratio = 0.5/1 |
| K306 (SATA) | 50 mol % LIP003; 27.5% cholesterol; 22 |
| | mol % DSPC; 0.5 mol % Maleimide-PEG- |
| | DSPE, RG7/Maleimide molar ratio = 0.02/1, |
| | CD19 antibody/RG7 molar ratio = 0.5/1 |
| K357 (DBCO/azide) | 50 mol % LIP003; 27.5% cholesterol; 22 |
| | mol % DSPC; 0.5 mol % Maleimide-PEG- |
| | DSPE, RG7/Maleimide molar ratio = 0.26/1, |
| | CD19 antibody/RG7 molar ratio = 0.5/1 |
| K358 (DBCO/Azide) | 50 mol % LIP003; 27.5% cholesterol; 22 |
| | mol % DSPC; 0.5 mol % Maleimide-PEG- |
| | DSPE, RG7/Maleimide molar ratio = 0.13/1, |
| | CD19 antibody/RG7 molar ratio = 0.5/1 |

Exemplary disease that can be treated by the disclosed LNPs include but are not limited to interstitial lung disease (e.g., pulmonary fibrosis, pnemonitis, histiocytosis, alveolar proteinosis, pulmonary hemosiderosis, alveolar microlithiasis, etc.) chronic obstructive pulmonary disease (COPD); chronic bronchitis; asthma; bronchiectasis; emphysema; lung cancer; cystic fibrosis; surfactant protein b (sp-b) deficiency; lower respiratory infections (influenza, pneumonia, acute bronchitis, etc.); pleural effusion. In some embodiments, disclosed LNPs can be used to treat a subject suffering from hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas. In some embodiments, disclosed LNPs can be used to treat a subject suffering from autoimmune disorders, including but not limited to rheumatoid arthritis, multiple sclerosis, type I diabetes, Addison disease, celiac disease, dermatomyositis, Graves disease, Hashimoto thyroiditis, myasthenia gravis, pernicious anemia, reactive arthritis, Sjogren syndrome, and systemic lupus erythematosus. In some embodiments, disclosed LNPs can be used to treat a subject suffering from an infectious disease, including but not limited to, Acute Flaccid Myelitis (AFM), Anaplasmosis, Anthrax, Avian Influenza, Babesiosis, Botulism, Brucellosis, Campylobacteriosis, Carbapenem-resistant Infection (CRE/CRPA), Chancroid, Chikungunya Virus Infection (Chikungunya), Chlamydia, Ciguatera (Harmful Algae Blooms (HABs)), Clostridium Difficile Infection, Clostridium Perfringens (Epsilon Toxin), Coccidioidomycosis fungal infection (Valley fever), COVID-19 (Coronavirus Disease 2019) COVID-19 (Coronavirus Disease 2019), Creutzfeldt-Jacob Disease, transmissible spongiform encephalopathy (CJD), Cryptosporidiosis (Crypto), Cyclosporiasis Dengue, 1,2,3,4 (Dengue Fever), Diphtheria, E. coli infection, Shiga toxin-producing (STEC), Eastern Equine Encephalitis (EEE), Ebola Hemorrhagic Fever (Ebola), Ehrlichiosis Encephalitis, Arboviral or parainfectious Enterovirus Infection, Non-Polio (Non-Polio Enterovirus), Enterovirus Infection, D68 (EV-D68), Giardiasis (Giardia), Glanders, Gonococcal Infection (Gonorrhea), Granuloma inguinale, Haemophilus Influenza disease, Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster, zoster VZV (Shingles), Histoplasmosis infection (Histoplasmosis), Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease Lymphogranuloma venereum infection (LGV), Malaria, Measles, Melioidosis, Meningitis, Viral (Meningitis, viral), Meningococcal Disease, Bacterial (Meningitis, bacterial), Middle East Respiratory Syndrome Coronavirus (MERS-COV), Monkeypox, Multisystem Inflammatory Syndrome in Children (MIS-C), Mumps, Norovirus, Pelvic Inflammatory Disease (PID), Pertussis (Whooping Cough), Plague; Bubonic, Septicemic, Pneumonic (Plague), Pneumococcal Disease (Pneumonia), Poliomyelitis (Polio), Powassan Psittacosis (Parrot Fever), Pustular Rash diseases (Small pox, monkeypox, cowpox), Q-Fever, Rabies, Ricin Poisoning, Rickettsiosis (Rocky Mountain Spotted Fever), Rubella, Including congenital (German Measles), Salmonellosis gastroenteritis (Salmonella), Scombroid Septic Shock (Sepsis), Severe Acute Respiratory Syndrome (SARS), Shigellosis gastroenteritis (Shigella), Smallpox, Staphyloccal Infection, Methicillin-resistant (MRSA), Staphylococcal Food Poisoning, Enterotoxin-B Poisoning (Staph Food Poisoning), Staphylococcal Infection, Vancomycin Intermediate (VISA), Staphylococcal Infection, Vancomycin Resistant (VRSA), Streptococcal Disease, Group A (invasive) (Strep A (invasive)), Streptococcal Disease, Group B (Strep-B), Streptococcal Toxic-Shock Syndrome, STSS, Toxic Shock (STSS, TSS), Syphilis, primary, secondary, early latent, late latent, congenital, Tetanus Infection, tetani (Lock Jaw), Trichomoniasis (Trichomonas infection), Trichonosis Infection (Trichinosis), Tuberculosis (TB), Tuberculosis (Latent) (LTBI), Tularemia (Rabbit fever), Typhoid Fever, Group D, Typhus Vaginosis, bacterial (Yeast Infection), Varicella (Chickenpox), Vibrio cholerae (Cholera), Vibriosis (Vibrio), Viral Hemorrhagic Fever (Ebola, Lassa, Marburg), West Nile Virus Yellow Fever, Yersenia (Yersinia), Zika Virus Infection.

In some embodiments, LNP used to treat a subject suffering from a disease can be administered as combination therapy. In some embodiments, an LNP used to treat a subject suffering from a disease can be administered to a subject in combination with other methods of treatment (e.g., standard of care treatment) for the disease.

In some embodiments LNPs of the present disclosure are evaluated for potency, selectivity, and/or tolerability. In some embodiments, potency is measured by level of mRNA expression in a specific targeted tissue (e.g., tumor, B-cell, lung). In some embodiments, selectivity is measured by ratio of mRNA expression in a specific targeted tissue relative to another tissue or a control tissue (e.g., lung/liver). In some embodiments, potency and selectivity are evaluated through in vivo and/or ex vivo measurements of mRNA expression. In some embodiments, potency and selectivity are evaluated through in vivo and/or ex vivo measurements of expression of the protein encoded by an mRNA.

In some embodiments, tolerability is evaluated with respect to common markers of immunogenicity, complement activation, and liver toxicity. These markers may include, but are not limited to, IFNg, IFNa, IL-1b, IL-6, MCP-1, TNFa, IP-10, sC5b-9, C3a, ALT, and AST.

### Methods of Manufacture

Methods of manufacturing lipid nanoparticles are known in the art. In one embodiment, the disclosed lipid nanoparticles are manufactured using microfluidics. For exemplary methods of using microfluidics to form lipid nanoparticles, see Leung, A. K. K, et al., J Phys Chem, 116:18440-18450 (2012), Chen, D., et al., J Am Chem Soc, 134:6947-6951 (2012), and Belliveau, N. M., et al., Molecular Therapy-Nucleic Acids, 1: e37 (2012). Briefly, the payload, such as a nucleic acid, is prepared in one buffer. The other lipid nanoparticle components (e.g., a first cationic ionizable lipid; a second cationic ionizable lipid; a sterol; a helper lipid; and a PEGylated lipid) are prepared in another (e.g., a second) buffer. A syringe pump introduces the two solutions into a microfluidic device. The two solutions come into contact within the microfluidic device to form lipid nanoparticles encapsulating the cargo.

In some embodiments, an LNP of the present disclosure is provided in a pharmaceutical composition. In some embodiments, the present disclosure provides a pharmaceutical composition comprising an LNP as described herein (e.g., and LNP comprising a nucleic acid) and excipients or accessory ingredients. Pharmaceutical compositions, LNP formulations and method of administration of LNPs are known in the art. Some formulations and methods of administration are described in, for example, WO2012135805 and WO201711286 each of which are incorporated herein in their entirety. In addition, techniques for formulation and administration of LNPs may be found in “Remington's Pharmaceutical Sciences” Mack Publishing Co, Easton, Pa.latest edition.

In some embodiments, a PEG-ligand is introduced to an LNP. In some embodiments, a PEG-ligand is prepared in a third buffer. In some embodiments, a PEG-ligand is incubated with a post-mixed LNP. In some embodiments, a PEG-ligand is incubated with a post-mixed LNP for 0.5 1, 1.5, 2, 2.5 hrs.

In some embodiments, antibody or antibody fragments are conjugated to an LNP. In some embodiments, prior to interacting with an LNP, an antibody or antibody fragment is modified to expose the functional groups in the buffer for 1, 2, 4, and 24 h hrs. In some embodiments, antibody or antibody fragments are purified by de-salting or dialysis. In some embodiments, LNPs are then incubated with antibody or antibody fragments in the buffer for 2, 4, or 24 h. In some embodiments, unconjugated antibody or antibody fragments are removed by gel filtration. Optionally, the targeted antibodies are incubated with monoclonal antibody-conjugated LNP for 0.5 h.

In some embodiments, a method for manufacturing the functionalized LNP is optimized to achieve the best targeting efficiency. In some embodiments, adjusted parameters include but are not limited to the ratio between the lipids in the lipid composition of the LNP core, the number of the functional groups per antibody or antibody fragment, the ratio of antibody or antibody fragment to PEG-derivatized lipids, the ratio of targeting antibody to antibody or antibody fragments, the time length of various steps, the purification process, or any combination thereof.

### EXEMPLIFICATION

### Example 1: Lipid Nanoparticle Delivery of RNA to Lung Tissue

The present example demonstrates utility of nucleic acid lipid particles of particular compositions. Specifically, the present example demonstrates the ability of lipid particles to be targeted to lung tissue based on the composition of the lipid nanoparticle.

Recently, Cheng et al. (Nat. Nanotechnol. 15, 313-320 (2020)) have demonstrated that the addition of the cationic lipid, DOTAP, to LNPs in a 50:50 molar ratio (DOTAP: sum of all other LNP components) yields potent and selective delivery of mRNA to the mouse lung. The goal of this study was to evaluate the cationic lipid DOTMA and the cationic ionizable lipid DC-Cholesterol as substitutes for DOTAP in the lung-targeting formulation. The present study demonstrates delivery of luciferase mRNA to mouse lung in vivo upon systemic administration of lipid nanoparticles (LNPs).

### Materials

LIP001 (MC3) was purchased from Nanosoft Polymer (Winston-Salem, USA). Cholesterol was purchased from Millipore Sigma (Burlington, USA). DSPC, PEG-DMG LIP027 (DOTAP), LIP028 (DOTMA), and LIP005 (DC-Cholesterol) were purchased from Avanti Polar lipids Inc. (Alabaster, USA).

### Formulation

Luciferase mRNA was formulated in LNPs containing a cationic ionizable lipid LIP001, cholesterol, DSPC, and PEG-DMG, in the presence or absence of the cationic lipid or cationic ionizable lipid pKa>7 according to the following molar ratios:

| | | | Exemplary Cationic | | | | |
|---|---|---|---|---|---|---|---|
| | | | Ionizable Lipid > | Exemplary Cationic | | | |
| | | | 7 pKa (e.g., DC- | Ionizable Lipid < | | | |
| | DOTAP | DOTMA | cholesterol) | 7 pKa (LIP001) | Cholesterol | DSPC | PEG-DMG |
| Formulation | (mol %) | (mol %) | (mol %) | (mol %) | (mol %) | (mol % | (mol %) |
| K005 | 0 | 0 | 0 | 50 | 38.5 | 10 | 1.5 |
| K004 | 50 | 0 | 0 | 25 | 19.25 | 5 | 0.75 |
| K021 | 0 | 50 | 0 | 25 | 19.25 | 5 | 0.75 |
| K020 | 0 | 0 | 50 | 25 | 19.25 | 5 | 0.75 |

The ratio of the sum of ionizable amines and cationic quaternary ammonium ions to mRNA phosphates was 6. Lipids were dissolved in ethanol and mRNA was dissolved in 50 mM Citrate buffer pH 5.0. The organic and aqueous phases were combined at a flow rate of 12 mL/min on a Precision Nanosystems NanoAssemblr at a ratio of 3:1 (aqueous: organic). The product was immediately diluted to 16.5% ethanol using MilliQ water. The diluted product was dialyzed against a volume of 50 mM Tris pH 7.5 45 mM NaCl that was 200-fold in excess. The dialyzed product was concentrated using Amicon Ultra 100 kDa centrifuge tubes and sterile filtered. mRNA concentration and encapsulation was assessed by the Invitrogen Ribogreen assay.

### In Vivo Luciferase Quantitation

LNPs were administered to B6 albino mice via the tail vein at a dose of 0.3 mg/kg. After six hours bioluminescence was measured using an IVIS Spectrum imager. Mice were treated with 150 mg/kg luciferin substrate administered via intraperitoneal injection. Immediately following the in vivo image mice were euthanized and the lung, liver and spleen were harvested for ex vivo imaging.

### Results

FIG. 1 illustrates in vivo images of the treatment groups. Luminescence signal is primarily visible from the liver for K005 while the lungs are the dominant feature in K004 and K021. K020 shows signal from both the liver and lungs.

FIG. 2 depicts the ex vivo luminescence of the treatment groups. Luminescence signal is primarily visible from the liver for K005 while the lungs are the dominant feature in K004 and K021. K020 shows signal from both the liver and lungs.

FIG. 3 demonstrates the ex vivo quantitation of bioluminescence for each LNP as well as mice treated with vehicle control. The data show that the addition of 50 mol % of either a cationic lipid or DC-cholesterol (K020) shifts the distribution of luminescence from liver to lung. The ratio of liver to lung luminescence is presented in Table 2.

TABLE 2

| | Liver:Lung |
|---|---|
| LNP | Luminescence |
| K005 | 144 |
| K004 | 0.029 |
| K021 | 0.028 |
| K020 | 3.63 |

The experiments presented here demonstrate that addition of cationic lipids DOTAP or DOTMA (50 mol %) to liver targeting LNPs shifts the distribution of luciferase expression to the lungs (˜5000-fold). The addition of the cationic ionizable lipid, DC Cholesterol, to liver targeting LNPs at 50 mol % shifts the distribution towards the lungs as well (˜40 fold).

### Example 2: Lipid Nanoparticles with PEG-Ligand Lipid Delivery of RNA to Lung Tissue

The present example also demonstrates utility of nucleic acid lipid particles of particular compositions. Specifically, the present example demonstrates the ability of lipid particles, including those comprising PEG-ligand, to be targeted to lung tissue based on the composition of the lipid nanoparticle.

### Materials

LIP001 (MC3) was purchased from Nanosoft Polymer (Winston-Salem, USA). LIP003 (Lipid 5) was purchased from DC Chemicals (Shanghai, China). Cholesterol was purchased from Millipore Sigma (Burlington, USA). DSPC,PEG-DMG and LIP027 (DOTAP) were purchased from Avanti Polar lipids Inc. (Alabaster, USA). FA-PEG-DSPE was purchased from Ruixi Biotech (Xi′an, China).

### Formulation

Luciferase mRNA was formulated in LNPs containing a cationic ionizable lipid, cholesterol, DSPC, and PEG-DMG, FA-PEG-DSPE in the presence of the cationic lipid DOTAP according to the following molar ratios:

| | | | Cationic | | | | FA- |
|---|---|---|---|---|---|---|---|
| | | Cationic | Ionizable | | | PEG- | PEG- |
| | | Ionizable | Lipid < 7pKa | Cholesterol | DSPC | DMG | DSPE |
| Formulation | DOTAP | Lipid < 7 pKa | (mol %) | (mol %) | (mol %) | (mol %) | (mol %) |
| K004 | 50 | LIP001 | 25 | 19.25 | 5 | 0.75 | 0 |
| K056 | 50 | LIP001 | 25 | 18.75 | 5 | 0.75 | 0.5 |
| K057 | 50 | LIP001 | 25 | 19.15 | 5 | 0.75 | 0.1 |
| K058 | 50 | LIP001 | 25 | 19.2 | 5 | 0.75 | 0.05 |
| K059 | 50 | LIP003 | 25 | 19.25 | 5 | 0.75 | 0 |
| K099 | 50 | LIP003 | 25 | 18.5 | 5 | 0.5 | 1 |
| K100 | 48.2 | LIP003 | 24.1 | 17.8 | 4.8 | 4 | 1 |
| K101 | 45.7 | LIP003 | 22.8 | 16.9 | 4.6 | 9 | 1 |
| K102 | 43.1 | LIP003 | 21.6 | 16 | 4.3 | 14 | 1 |

The ratio of the sum of ionizable amines and cationic quaternary ammonium ions to mRNA phosphates was 6. Lipids were dissolved in ethanol and mRNA was dissolved in 50 mM Citrate buffer pH 5.0. The organic and aqueous phases were combined at a flow rate of 12 mL/min on a Precision Nanosystems NanoAssemblr at a ratio of 3:1 (aqueous: organic). The product was immediately diluted to 16.5% ethanol using MilliQ water. If included, PEG-ligand was introduced to the LNPs prior to dialysis. PEG-ligand was solubilized in a buffer of 50 mM citrate pH 5.0 containing 16.5% ethanol. PEG-ligand was introduced at a concentration of approximately 2.5 mg/mL and was allowed to incubate with the post-mixed LNPs for approximately 1 hour. The diluted product was dialyzed against a volume of 50 mM Tris pH 7.5 45 mM NaCl that was 200-fold in excess. The dialyzed product was concentrated using Amicon Ultra 100 kDa centrifuge tubes and sterile filtered. mRNA concentration and encapsulation was assessed by the Invitrogen Ribogreen assay. LIP003 is described in Sabnis et al., Mol. Ther. 26 (6) 1509-1519 (2018); and the structure is provided in Table 4.

### In Vivo Luciferase Quantitation

LNPs were administered to B6 albino mice via the tail vein at a dose of 0.3 mg/kg. After eighteen hours bioluminescence was measured using an IVIS Spectrum imager. Mice were treated with 150 mg/kg luciferin substrate administered via intraperitoneal injection. Immediately following the in vivo image mice were euthanized and the lung, liver and spleen were harvested for ex vivo imaging.

### Results

FIG. 4 depicts the ex vivo quantitation of lung bioluminescence 18 hours after dosing of the indicated LNPs. The results demonstrate a 3-fold, 4-fold, and 5-fold increase in lung bioluminescence for the addition of PEG ligand at 0.5, 0.1, and 0.05 mol %, respectively.

FIG. 5 illustrates that the exchange of cationic ionizable lipid LIP001 for cationic ionizable lipid LIP003 does not alter the lung specificity of the lipid nanoparticle.

FIG. 6 illustrates the impact of additional PEG lipid added to the formulation. The addition of PEG-lipid reduces the expression of luciferase in the lungs in a dose dependent manner.

### Example 3: Evaluation of Alternative Lipids Included in Lipid Nanoparticles

The present example provides further demonstration of the utility of nucleic acid lipid particles of particular compositions as described herein to target particular tissues or cells.

Nucleic acid lipid particles are formulated with luciferase mRNA in LNPs comprising DOTAP or one or more cationic ionizable lipid(s) with pKa>7 to assess potency (level of mRNA expression in the lung), selectivity (the ratio of expression in the lung and liver) and tolerability. Potency and selectivity are evaluated through in vivo and ex vivo measurement of luciferase in mice and specific organs using an IVIS imaging system and luciferin substrate. Tolerability is evaluated with respect to common markers of immunogenicity, complement activation, and liver toxicity. These markers may include, but are not limited to, IFNg, IFNa, IL-1b, IL-6, MCP-1, TNFa, IP-10, sC5b-9, C3a, ALT, and AST.

Nucleic acid lipid particles are formulated with luciferase mRNA in LNPs comprising one or more cationic ionizable lipid(s) with pKa<7 to assess potency (level of mRNA expression in the lung), selectivity (the ratio of expression in the lung and liver) and tolerability. Potency and selectivity are evaluated through in vivo and ex vivo measurement of luciferase in mice and specific organs using an IVIS imaging system and luciferin substrate. Tolerability is evaluated with respect to common markers of immunogenicity, complement activation, and liver toxicity. These markers may include, but are not limited to, IFNg, IFNa, IL-1b, IL-6, MCP-1, TNFa, IP-10, sC5b-9, C3a, ALT, and AST.

Nucleic acid lipid particles are formulated with mRNA in LNPs comprising one or more PEG-lipid-ligands. Conjugated ligands (i.e., ligands in a PEG-lipid-ligand) may include, but are not limited to, antibodies targeting cell surface proteins, hyaluronic acid, small molecules, peptides, and peptides that target integrins. LNPs comprising one or more PEG-lipid-ligands and one or more cationic ionizable lipid(s) are formulated and assessed for potency (level of mRNA expression in the lung), selectivity (the ratio of expression in the lung and liver) and tolerability. Potency and selectivity are evaluated through in vivo and ex vivo measurement of luciferase in mice and specific organs using an IVIS imaging system and luciferin substrate. Tolerability is evaluated with respect to common markers of immunogenicity, complement activation, and liver toxicity. These markers may include, but are not limited to, IFNg, IFNa, IL-1b, IL-6, MCP-1, TNFa, IP-10, sC5b-9, C3a, ALT, and AST.

A Design of Experiments approach is used to explore the multidimensional space of lipid components and to perform compositional optimization. Various lipid compositional ranges are tested as listed in the following table:

TABLE 3

| Component | Low (mol %) | High (mol %) |
|---|---|---|
| Cationic Ionizable Lipid | 0 | 60 |
| (pKa < 7) | | |
| Cationic Ionizable Lipid | 0 | 80 |
| (pKa > 7) | | |
| Cationic Ionizable Lipid | 0 | 60 |
| (pKa < 7) | | |
| Cationic Ionizable Lipid | 0 | 80 |
| (pKa > 7) | | |
| Sterol | 0 | 40 |
| Phospholipid | 0 | 20 |
| Peg Lipid | 0 | 25 |
| Peg Lipid Ligand | 0 | 25 |

The various LNP compositions are formulated with luciferase mRNA to assess potency (level of mRNA expression in the lung), selectivity (the ratio of expression in the lung and liver) and tolerability. Potency and selectivity are evaluated through in vivo and ex vivo measurement of luciferase in mice and specific organs using an IVIS imaging system and luciferin substrate. Tolerability is evaluated with respect to common markers of immunogenicity, complement activation, and liver toxicity. These markers may include, but are not limited to, IFNg, IFNa, IL-1b, IL-6, MCP-1, TNFa, IP-10, sC5b-9, C3a, ALT, and AST.

Flow cytometry is used to perform single cell analysis on protein expression in digested lung tissue to determine which cell types are being transfected. Briefly, mRNA encoding a fluorescent protein is formulated in lead formulation candidates determined by previously described experiments. The LNPS are introduced into mice. 24 hours post dosing, lungs are harvested and digested into single cell suspensions which are labeled with cell-type-specific fluorescently conjugated antibodies. A flow cytometer will be used to quantify the amount of expressed protein per cell type. Additionally, this flow cytometry approach is used as an optimization parameter when performing formulation screens as described above.

### Example 4: Additional Lipid Nanoparticles with Lipid Delivery of RNA to Lung Tissue

The present example provides further demonstration of utility of nucleic acid lipid particles of particular compositions. Specifically, the present example demonstrates the ability of lipid particles, comprising alternative cationic ionizable lipid pKa>7, to be targeted to lung tissue based on the composition of the lipid nanoparticle.

### Materials

LIP001 (MC3) was purchased from Nanosoft Polymer (Winston-Salem, USA). LIP003 (Lipid 5) was purchased from DC Chemicals (Shanghai, China). Cholesterol was purchased from Millipore Sigma (Burlington, USA). DSPC and PEG-DMG were purchased from Avanti Polar lipids Inc. (Alabaster, USA). LIP019 (DLin-M-C3-MA), LIP020, LIP021 and LIP22 (ALYN-139) were purchased from Aragen (Hyderabad, India).

### Formulation

Luciferase mRNA was formulated in LNPs containing a cationic ionizable lipid <7 pKa LIP003, cholesterol, DSPC, and PEG-DMG, in the presence of the cationic ionizable lipid >7 pKa according to the following molar ratios:

| | | Cationic | | Cationic | | | |
|---|---|---|---|---|---|---|---|
| | Cationic | Ionizable | Cationic | Ionizable | | | PEG- |
| | Ionizable | Lipid < 7 pK | Ionizable | Lipid > 7 pK | Cholesterol | DSPC | DMG |
| Formulation | Lipid < 7 pK | (mol %) | Lipid > 7 pK | (mol %) | (mol %) | (mol %) | (mol %) |
| K261 | LIP003 | 25 | LIP019 | 50 | 19.25 | 5 | 0.75 |
| K262 | LIP003 | 25 | LIP020 | 50 | 19.25 | 5 | 0.75 |
| K263 | LIP003 | 25 | LIP021 | 50 | 19.25 | 5 | 0.75 |
| K264 | LIP003 | 25 | LIP022 | 50 | 19.25 | 5 | 0.75 |

The ratio of the sum of ionizable amines and cationic quaternary ammonium ions to mRNA phosphates in the LNP was 6. Lipids were dissolved in ethanol and mRNA was dissolved in 50 mM Citrate buffer pH 5.0. The organic and aqueous phases were combined at a flow rate of 12 mL/min on a Precision Nanosystems NanoAssemblr at a ratio of 3:1 (aqueous: organic). The product was immediately diluted to 16.5% ethanol using MilliQ water. The diluted product was dialyzed against a volume of 50 mM Tris pH 7.5 45 mM NaCl that was 300-fold in excess. The dialyzed product was concentrated using Amicon Ultra 100 kDa centrifuge tubes and sterile filtered. mRNA concentration and encapsulation was assessed by the Invitrogen Ribogreen assay.

### In Vivo Luciferase Quantitation

LNPs were administered to BALB/c mice via the tail vein at a dose of 0.5 mg/kg. After eighteen hours bioluminescence was measured using an IVIS Spectrum imager. Mice were treated with 150 mg/kg luciferin substrate administered via intraperitoneal injection. Immediately following the in vivo image mice were euthanized and the lung, liver and spleen were harvested for ex vivo imaging.

### Results

FIG. 7 illustrates in vivo images of bioluminescence 18 hours after dosing of the indicated LNPs. FIG. 8 depicts the ex vivo luminescence of the treatment groups. FIG. 9 demonstrates the ex vivo quantitation of bioluminescence for each LNP. Notably, K263 containing lipid LIP021 has lung targeting with a low liver/lung ratio. Surprisingly, K262 containing lipid LIP020 exhibits spleen targeting with a low liver/spleen ratio.

To further test the lung targeting efficacy of LNPs comprising lipid LIP021, LNPs comprising cationic ionizable lipid <7 pKa LIP001 instead of LIP005 were produced according to the following molar ratios. LNPs K271 comprising LIP021 without any cationic ionizable lipid <7 pKa were also produced to test the sole effect of LIP021:

| | | Cationic | | Cationic | | | |
|---|---|---|---|---|---|---|---|
| | Cationic | Ionizable | Cationic | Ionizable | | | PEG- |
| | Ionizable | Lipid < 7 pK | Ionizable | Lipid > 7 pK | Cholesterol | DSPC | DMG |
| Formulation | Lipid < 7 pK | (mol %) | Lipid > 7 pK | (mol %) | (mol %) | (mol %) | (mol %) |
| K269 | LIP001 | 25 | LIP021 | 50 | 19.25 | 5 | 0.75 |
| K271 | | | LIP021 | 50 | 38.5 | 10 | 1.5 |

FIG. 10 demonstrates the ex vivo quantitation of bioluminescence for each LNP. FIG. 11 demonstrates the ex vivo quantitation of bioluminescence for each LNP. Notably, the exchange of cationic ionizable lipid LIP003 for cationic ionizable lipid LIP001 does not alter the lung specificity of the lipid nanoparticle. LIP021 itself, without combining other cationic ionizable lipids, does not exhibit a lung-targeting effect.

### Example 5: Lipid Nanoparticles Useful for Delivery to Extrahepatic Tissues

The present example demonstrates utility of nucleic acid lipid particles of particular compositions. Specifically, the present example demonstrates the ability of lipid particles to deliver nucleic acids to tissues other than the liver based on the composition of the lipid nanoparticle.

LIP002 (LP-01) was purchased from Medkoo Biosciences (Morrisville, USA). LIP003 (Lipid 5) was purchased from DC Chemicals (Shanghai, China). Cholesterol was purchased from Millipore Sigma (Burlington, USA). DSPC, PEG-DMG, PEG-DSG and PEG-DSPE were purchased from Avanti Polar lipids Inc. (Alabaster, USA). FA-PEG-DSPE was purchased from Ruixi Biotech (Xi′an, China).

### Formulation

Luciferase mRNA was formulated in LNPs according to the following molar ratios:

| | | Cationic | | | | |
|---|---|---|---|---|---|---|
| | Cationic | Ionizable | | | | PEG |
| | Ionizable | Lipid < 7pK | Cholesterol | DSPC | PEG | Lipid |
| Formulation | Lipid < 7 pK | (mol %) | (mol %) | (mol %) | Lipid | (mol %) |
| K005 | LIP003 | 50 | 38.5 | 10 | PEG- | 1.5 |
| | | | | | DMG | |
| K023 | LIP003 | 50 | 38.5 | 10 | PEG- | 1.5 |
| | | | | | DSPE | |
| K024 | LIP003 | 50 | 39.8 | 10 | PEG- | 0.2 |
| | | | | | DSPE | |
| K029 | LIP002 | 50 | 27.5 | 22 | PEG- | 0.5 |
| | | | | | DMG | |
| K030 | LIP003 | 50 | 27.5 | 22 | PEG- | 0.5 |
| | | | | | DMG | |

The ratio of the cationic ionizable amines to mRNA phosphates in the LNP was 6. Lipids were dissolved in ethanol and mRNA was dissolved in 50 mM Citrate buffer pH 5.0. The organic and aqueous phases were combined at a flow rate of 12 mL/min on a Precision Nanosystems. NanoAssemblr at a ratio of 3:1 (aqueous: organic). The product was immediately diluted to 16.5% ethanol using MilliQ water. The diluted product was dialyzed against a volume of 50 mM Tris pH 7.5 45 mM NaCl that was 300-fold in excess. The dialyzed product was concentrated using Amicon Ultra 100 kDa centrifuge tubes and sterile filtered. mRNA concentration and encapsulation were assessed by the Invitrogen Ribogreen assay.

### In Vivo Luciferase Quantitation

BALB/c mice were s.c. inoculated with 2 million B16-F10 or 5 million A20 cells at the right flank. When tumor size reached ˜300-500 mm³ (10 days post inoculation), LNPs were administered to B16-F10-bearing mice via the tail vein at a dose of 0.3, 1 or 3 mg/kg. After eighteen hours, bioluminescence was measured using an IVIS Spectrum imager. Mice were treated with 150 mg/kg luciferin substrate administered via intraperitoneal injection. Immediately following the in vivo image mice were euthanized and the tumor and liver were harvested for ex vivo imaging. Luminescence was quantitated using Aura software. Regions of Interest (ROIs) were drawn around tumors. The flux and area of the ROIs was recorded. The areas were used to normalize the flux to volume assuming that tumors were spherical.

### Results

FIG. 12 illustrates in vivo images of the treatment groups. Luminescence signal of LNPs K029 and K030 are distributed to tissues other than the liver whereas LNPs K005 are concentrated in the liver. FIG. 13(A-D) demonstrate quantification of luminescence. FIG. 13 C demonstrates LNPs of the present disclosure, particularly K029 and K030, concentrate in tissues other than the liver. Moreover, as demonstrated by FIG. 13D LNPs of the present disclosure, particularly K030, can concentrate in B16-F10 solid tumors.

Additional lipid nanoparticle formulations were tested to identify LNP formulations that concentrate delivery of mRNA to tumors. Luciferase mRNA was formulated in LNPs according to the following molar ratios:

| Formula- | LIP003 | Cholesterol | DSPC | | PEG lipid |
|---|---|---|---|---|---|
| tion | (mol %) | (mol %) | (%) | PEG Lipid | (mol %) |
| K087 | 50 | 39.5 | 10 | PEG-DSPE | 0.5 |
| K104 | 50 | 38.5 | 10 | PEG-DSG | 1.5 |
| K105 | 50 | 37 | 10 | PEG-DSG | 3 |
| K106 | 50 | 35 | 10 | PEG-DSG | 5 |
| K107 | 50 | 37 | 10 | FA-PEG-DSPE | 3 |
| K108 | 50 | 27.5 | 22 | PEG-DSPE | 0.5 |
| K103 | 50 | 39.5 | 10 | PEG-DSG | 0.5 |
| K110 | 50 | 23 | 22 | PEG-DSG | 5 |
| K111 | 50 | 25 | 22 | FA-PEG-DSPE | 3 |

FIG. 14 demonstrates the distribution of the LNPs in whole body, liver, and tumor. Particularly, LNPs K108 and K111 express in B16-F10 tumors to a greater extent than other tested LNPs at 0.3 mg RNA/kg. FIG. 15 demonstrates that K087, K108, and K111 express in B16-F10 tumors at least 10 times greater than the K005 LNP at 0.3 mg mRNA/kg. Increasing doses to 3 mg/kg further boosted the mRNA expression tumor. FIG. 16 demonstrates that K087, K108, and K111 express in A20 tumors at least 5 times greater than the K005 LNP at 1 mg mRNA/kg.

FIG. 17 demonstrates additional LNP nucleic acid formulations which concentrate in tissues other than the liver. Luciferase mRNA was formulated in LNPs according to the following molar ratios:

| | | | | PEG- |
|---|---|---|---|---|
| | LIP003 | Cholesterol | DSPC | DMG |
| Formulation | (mol %) | (mol %) | (mol %) | (mol %) |
| K048 | 58.7 | 24.9 | 15.9 | 0.5 |
| K049 | 34.8 | 48.8 | 15.9 | 0.5 |
| K050 | 34.8 | 24.9 | 39.8 | 0.5 |
| K051 | 46.8 | 36.8 | 15.9 | 0.5 |
| K052 | 46.8 | 24.9 | 27.9 | 0.5 |
| K053 | 34.8 | 36.8 | 27.9 | 0.5 |
| K054 | 42.8 | 32.8 | 23.9 | 0.5 |

Notably, formulations K051; K052; K053, and K054 show a low liver to whole body ratio.

### Example 6: Surface Functionalized LNPS Using Standard LNP Core for Cell Targeting

The present example demonstrates utility of nucleic acid lipid particles of particular compositions. Specifically, the present example demonstrates the ability of lipid particles, including those comprising antibody surface functionalization on the standard LNP core, to deliver nucleic acids to specific cell types based on the composition of the lipid nanoparticle.

### Materials

LIP003 (Lipid 5) was purchased from DC Chemicals (Shanghai, China). Cholesterol was purchased from Millipore Sigma (Burlington, USA). DSPC, PEG-DMG, Maleimide-PEG-DSPE were purchased from Avanti Polar lipids Inc. (Alabaster, USA). Mouse anti-rat IgG2a (clone RG7/1.30), rat anti-mouse CD19 mAb (clone 1D3) and rat IgG2a isotype control (clone 2A3) were purchased from Bio X Cell (Lebanon, USA). DTT and EDTA were purchased from Thermo Scientific (Waltham, USA). DiD was purchased from Invitrogen (Waltham, USA).

### Formulation

### Using DTT to Introduce Thio to Antibody and Maleimide/Thio Chemistry to Conjugate Antibody to LNPs

FIG. 18 demonstrates an exemplary process for conjugating antibodies to the surface of an LNP. Step 1: The ratio of cationic ionizable amines to mRNA phosphates in the LNP was 6. Lipids were dissolved in ethanol and mRNA was dissolved in 50 mM Citrate buffer pH 5.0. The organic and aqueous phases were combined at a flow rate of 12 mL/min on a Precision Nanosystems NanoAssemblr at a ratio of 3:1 (aqueous: organic). The product was immediately diluted to 16.5% ethanol using MilliQ water. The diluted product was dialyzed against a volume of 1×PBS that was 300-fold in excess. The dialyzed product was concentrated using Amicon Ultra 100 kDa centrifuge tubes and sterile filtered. mRNA concentration and encapsulation were assessed by the Invitrogen Ribogreen assay. Step 2: Anti-IgG secondary antibody (RG7) was reduced in 1×PBS containing 1 mM dithiothreitol (DTT) and 5 mM EDTA for 1 hour at room temperature. The reduced RG7 was then purified by buffer exchange to 1×PBS with 5 mM EDTA using 7K Zeba spin desalting column to remove excess DTT. The reduced secondary antibody was quantified by Nanodrop. Step 3: The reduced secondary antibody was added to LNP at various rations of RG7/maleimide ratios and incubated for 2 hours at room temperature. Step 4: RG7 conjugated LNPs were separated from unconjugated RG7 using Sepharose CL4B beads on a gel filtration column with PBS as the mobile phase. Fractions containing pure LNPs were identified by the fluorescent signal of DiD-loaded in LNPs, and then were pooled together. mRNA concentration and encapsulation were assessed by the Invitrogen Ribogreen assay. RG7 concentration was assessed by the Pierce BCA protein assay kit. Step 5: The targeting (primary) antibody was added to RG7-LNPs at various of primary/secondary weight or molar ratios and incubated for 30 min at room temperature. Step 6 (optional): The targeting antibody bound LNPs were separated from unbound targeting antibody using Sepharose CL4B beads on a gel filtration column with PBS as the mobile phase. Fractions containing pure LNPs were identified by fluorescent signal of DiD-loaded in LNPs, and then were pooled together. mRNA concentration and encapsulation were assessed by the Invitrogen Ribogreen assay.

Luciferase mRNA was formulated in LNPs containing a cationic ionizable lipid LIP003, cholesterol, DSPC, PEG-DMG, maleimide-PEG-DSPE, secondary antibody RG7 and primary antibody (CD19 mAb or isotype IgG) according to the following molar ratios:

| | | | | | Maleimide- | | | |
|---|---|---|---|---|---|---|---|---|
| | LIP003 | Cholesterol | DSPC | PEG-DMG | PEG-DSPE | RG7/Maleimide | Primary | Primary/RG7 |
| Formulation | (mol %) | (mol %) | mol %) | (mol %) | (mol %) | molar ratio | Antibody | weight ratio |
| K219 | 50 | 38 | 10 | 1.5 | | | | |
| K226 | 50 | 39.5 | 10 | 0.5 | | | | |
| K227 | 50 | 38 | 10 | 1.5 | 0.5 | 0.32 | CD19 | 0.1 |
| K228 | 50 | 38 | 10 | 1.5 | 0.5 | 0.32 | ISO | 0.1 |
| K229 | 50 | 36.5 | 10 | 3 | 0.5 | 0.32 | CD19 | 0.1 |
| K230 | 50 | 36.5 | 10 | 3 | 0.5 | 0.32 | ISO | 0.1 |
| K242 | 50 | 38.5 | 10 | 1 | 0.5 | 0.32 | ISO | 0.1 |
| K243 | 50 | 38.5 | 10 | 1.0 | 0.5 | 0.32 | CD19 | 0.1 |

### In Vitro Cell Binding Assay

A20 cells were treated with DiD loaded-LNPs at mRNA concentration of 2 μg/mL. After one hour, cells were washed with 1×PBS twice to remove unbound LNPs. A flow cytometer was used to quantify the DiD fluorescent intensity of treated A20 cells.

### In Vitro Luciferase Quantitation

A20 cells were treated with luciferase mRNA-loaded LNPs at mRNA concentration of 2 μg/mL. Cells were tested in three different media: OPTI-MEM, OPTI-MEM containing 5% mouse serum, or OPTI-MEM containing 0.8 ug/mL recombinant human ApoE4, USA. After twenty-four hours, OneGLOW reagent was applied to treated cells and the luminescence was measured using microplate reader.

### In Vivo Luciferase Quantitation

BALB/c mice were s.c. inoculated with 5 million A20 cells at the right flank. When tumor size reached ˜300-500 mm³ (10 days post inoculation), LNPs were administered to A20-bearing mice intravenously or intratumorally at a dose of 0.5 mg/kg. After eighteen hours bioluminescence was measured using an IVIS Spectrum imager. Mice were treated with 150 mg/kg luciferin substrate administered via intraperitoneal injection. Immediately following the in vivo image mice were euthanized and the tumor were harvested for ex vivo imaging. Luminescence was quantitated using Aura software. Regions of Interest (ROIs) were drawn around tumors. The flux and area of the ROIs was recorded. The areas were used to normalize the flux to volume assuming that tumors were spherical.

### Results

Various primary/secondary antibody ratios are tested in the conjugation procedure described herein to maximize the targeting efficiency. As shown in FIG. 19, A primary/secondary ratio of 0.1:1 demonstrates the highest MFI and % of DiD+ cells for CD19 antibody conjugated LNPs (K227 and K229) binding to A20 cells.

FIG. 20 demonstrates CD19-LNPs K227 and K243 has higher transfection efficiency than their ISO conjugated counterparts (K228 and K242 respectively) in vitro in OPTI-MEM and OPTI-MEM w/5% mouse serum. In addition, decreasing PEG-DMG from 1.5% (K227) to 1% (K243) further increases transfection efficiency in vitro. The higher transfection of CD19-LNPs than their ISO counterparts is abolished in OPTI-MEM containing ApoE4.

FIG. 21 showed all CD19-LNPs (K227, K229, and K243) using standard LNP core were worse than non-targeting LNPs (K219 and K226) and didn't show improvement over their ISO counterparts (K228, K230, and K242) in the expression of mRNA in A20 solid tumor model.

### Example 7: Surface Functionalized LNPS Using Extrahepatic LNP Core for Cell Targeting

The present example demonstrates utility of nucleic acid lipid particles of particular compositions. Specifically, the present example demonstrates the ability of lipid particles, including those comprising antibody surface functionalization on the extrahepatic LNP core, to deliver nucleic acids to specific cell types based on the composition of the lipid nanoparticle.

### Materials

LIP003 (Lipid 5) was purchased from DC Chemicals (Shanghai, China). Cholesterol was purchased from Millipore Sigma (Burlington, USA). DSPC and Maleimide-PEG-DSPE were purchased from Avanti Polar lipids Inc. (Alabaster, USA). Mouse anti-rat IgG2a (clone RG7/1.30), rat anti-mouse CD19 mAb (clone 1D3) and rat IgG2a isotype control (clone 2A3) were purchased from Bio X Cell (Lebanon, USA). DTT and EDTA was purchased from Thermo Scientific (Waltham, USA). DiD was purchased from Invitrogen (Waltham, USA).

### Formulation

mCherry mRNA was formulated in LNPs containing a cationic ionizable lipid LIP003, cholesterol, DSPC, maleimide-PEG-DSPE, secondary antibody RG7 and primary antibody (CD19 mAb or isotype IgG) according to the following molar ratios:

| | | | | | Maleimide- | | | | Purification |
|---|---|---|---|---|---|---|---|---|---|
| | LIP003 | Cholesterol | DSPC | PEG-DMG | PEG-DSPE | RG7/Maleimide | Primary | Primary/RG7 | post primary |
| Formulation | (mol %) | (mol %) | mol %) | (mol %) | (mol %) | molar ratio | Antibody | weight ratio | antibody binding |
| K247 | 50 | 38 | 10 | 1.5 | | | | | |
| K249 | 50 | 27.5 | 22 | 0 | 0.5 | | | | |
| K256 | 50 | 27.5 | 22 | 0 | 0.5 | 0.32 | | | |
| K257 | 50 | 27.5 | 22 | 0 | 0.5 | 0.32 | ISO | 0.1 | |
| K258 | 50 | 27.5 | 22 | 0 | 0.5 | 0.32 | CD19 | 0.1 | |
| K259 | 50 | 27.5 | 22 | 0 | 0.5 | 0.32 | CD19 | 1 | |
| K302 | 50 | 27.5 | 22 | 0 | 0.5 | 0.32 | ISO | 0.05 | |
| K303 | 50 | 27.5 | 22 | 0 | 0.5 | 0.32 | CD19 | 0.05 | |
| K304 | 50 | 38.5 | 10 | 1.0 | 0.5 | 0.32 | CD19 | 0.05 | Yes |
| K361 | 50 | 27.5 | 22 | 0 | 0.5 | 0.16 | CD19 | 0.1 | |
| K362 | 50 | 27.5 | 22 | 0 | 0.5 | 0.06 | CD19 | 0.5 | |

### In Vitro Transfection Efficiency Quantitation

A20 cells were treated with mCherry mRNA-loaded LNPs at mRNA concentration of 2 μg/mL. Cells were tested in three different mediums: OPTI-MEM, OPTI-MEM containing 5% mouse serum, or OPTI-MEM containing 0.8 ug/mL recombinant human ApoE4. After one hour, cells were washed with 1×PBS twice to remove unbound LNPs. A flow cytometer was used to quantify the DiD and mCherry fluorescent intensity of treated A20 cells.

### In Vivo Transfection Efficiency Quantitation

LNPs were administered to BALB/c mice intravenously at a dose of 0.5 mg/kg. After eighteen hours, multiple organs, including spleen, bone barrow and blood were harvested and processed into single cell suspension. Cells were then stained with Ghost dye in PBS for 30 min on ice, followed by surface marker staining including B220, and TER-119 in PEB for 30 min on ice. A flow cytometer was used to quantify the DiD and mCherry fluorescent intensity in B cells from treated mice.

### Results

FIG. 22 demonstrates that functionalizing extrahepatic targeting LNPs with CD19 antibodies ((K256-CD19) can increase targeting efficiency than its ISO counterparts (K256-ISO) and non-targeting LNPs (K247 and K249) in A20 cells in all three different mediums: OPTI-MEM, OPTI-MEM containing 5% mouse serum, or OPTI-MEM containing 0.8 ug/mL ApoE4. The optimal primary/secondary antibody weight ratio is 0.1:1, with the highest % of mCherry+ cells.

FIGS. 23 and 24 demonstrate that extrahepatic targeting LNPs functionalized with anti-CD19 antibodies (K258 and K259) have improved B cell targeting in the spleen and blood of naïve BALB/c mice ISO-LNPs (K257) and other non-targeting LNPs (K247, K249 and K256). Moreover, FIG. 25 demonstrates that extrahepatic targeting LNPs functionalized with anti-CD19 antibodies show no preferential binding to non-B cells.

An additional purification step after the primary antibody binding to the secondary antibody was also evaluated. FIG. 26 demonstrates LNP K304 with an additional washing step improves the targeting efficiency to B cells than K303 without a washing step in vivo and in vivo.

Various secondary antibody (RG7) to maleimide-PEG-DSPE rations were tested in the conjugation procedure described herein to maximize the targeting efficiency. FIG. 27 demonstrates LNP K362 with the reduced RG7/maleimide ratio improves the targeting efficacy in immature B cells.

### Example 8: Surface Functionalized LNPS Using Extrahepatic LNP Core for Cell Targeting Using Other Conjugation Chemistry

The present example demonstrates utility of nucleic acid lipid particles of particular compositions. Specifically, the present example demonstrates the ability of lipid particles, including those comprising antibody surface functionalization on the extrahepatic LNP core using alternative conjugation chemistry, to deliver nucleic acids to specific cell types based on different conjugation methods.

### Materials

LIP003 (Lipid 5) was purchased from DC Chemicals (Shanghai, China). Cholesterol was purchased from Millipore Sigma (Burlington, USA). DSPC, Maleimide-PEG-DSPE and Azide-PEG-DSPE were purchased from Avanti Polar lipids Inc. (Alabaster, USA). Mouse anti-rat IgG2a (clone RG7/1.30), rat anti-mouse CD19 mAb (clone 1D3) and rat IgG2a isotype control (clone 2A3) were purchased from Bio X Cell (Lebanon, USA). SATA ((N-succinimidyl S-acetylthioacetate) and EDTA were purchased from Thermo Scientific (Waltham, USA). DiD was purchased from Invitrogen (Waltham, USA). FAM-Azide 5 isomer was purchased from Lumiprobe (Cockeysville, USA). DBCO-PEG₄-NHS was purchased from BroadPharm (San Diego, USA).

### Formulation

### Using SATA to Introduce Thio to Antibody and Maleimide/Thio Chemistry to Conjugate Antibody to LNPs

Step 1: The ratio cationic ionizable amines to mRNA phosphates in the LNP was 6. Lipids were dissolved in ethanol and mRNA was dissolved in 50 mM Citrate buffer pH 5.0. The organic and aqueous phases were combined at a flow rate of 12 mL/min on a Precision Nanosystems NanoAssemblr at a ratio of 3:1 (aqueous: organic). The product was immediately diluted to 16.5% ethanol using MilliQ water. The diluted product was dialyzed against a volume of 1×PBS that was 300-fold in excess. The dialyzed product was concentrated using Amicon Ultra 100 kDa centrifuge tubes and sterile filtered. mRNA concentration and encapsulation was assessed by the Invitrogen Ribogreen assay. Step 2: Anti-IgG secondary antibody (RG7) was incubated with 10× molar excess of SATA using Pierce™ Sulfhydryl Addition Kit. The sulfhydryl modified-RG7 was then purified by buffer exchange to 1×PBS with 5 mM EDTA using 7K Zeba spin desalting column to remove excess SATA. The sulfhydryl-modified secondary antibody was quantified by Nanodrop. The sulfhydryl group per antibody was measured by Ellman's assay. Step 3: The sulfhydryl modified-RG7 was added to LNP at various rations of RG7/maleimide ratios and incubated for 2 hours at room temperature. Step 4: RG7 conjugated LNPs were separated from unconjugated RG7 using Sepharose CL4B beads on a gel filtration column with PBS as the mobile phase. Fractions containing pure LNPs were identified by fluorescent signal of DiD-loaded in LNPs, and then were pooled together. mRNA concentration and encapsulation were assessed by the Invitrogen Ribogreen assay. RG7 concentration was assessed by the Pierce BCA protein assay kit. Step 5: The targeting (primary) antibody was added to RG7-LNPs at various of primary/secondary weight or molar ratios and incubated for 30 min at room temperature. Step 6 (optional): The targeting antibody bound LNPs were separated from unbound targeting antibody using Sepharose CL4B beads on a gel filtration column with PBS as the mobile phase. Fractions containing pure LNPs were identified by fluorescent signal of DiD-loaded in LNPs, and then were pooled together. mRNA concentration and encapsulation were assessed by the Invitrogen Ribogreen assay.

mCherry mRNA was formulated in LNPs containing a cationic ionizable lipid LIP003, cholesterol, DSPC, PEG-DMG, maleimide-PEG-DSPE, secondary antibody RG7 and primary antibody (CD19 mAb or isotype IgG) according to the following molar ratios:

| | | | | | Maleimide- | | | | Purification |
|---|---|---|---|---|---|---|---|---|---|
| | LIP003 | Cholesterol | DSPC | PEG-DMG | PEG-DSPE | RG7/Maleimide | Primary | Primary/RG7 | Post Primary |
| Formulation | (mol %) | (mol %) | mol %) | (mol %) | (mol %) | molar ratio | Antibody | Molar Ratio | Antibody Binding |
| K296 | 50 | 27.5 | 22 | 0 | 0.5 | 0.1 | CD19 | | |
| K298 | 50 | 27.5 | 22 | 0 | 0.5 | 0.02 | CD19 | | |
| K300 | 50 | 27.5 | 22 | 0 | 0.5 | 0.04 | CD1 | | |
| | | | | | | | 9 | | |
| K305 | 50 | 27.5 | 22 | 0 | 0.5 | 0.02 | CD19 | 0.5 | |
| K306 | 50 | 27.5 | 22 | 0 | 0.5 | 0.02 | CD19 | 0.5 | Yes |

### Using DBCO-PEG₄-NHS to Introduce DBCO to Antibody and DBCO/Azide Chemistry to Conjugate Antibody to LNPs

Step 1: The ratio of cationic ionizable amines to mRNA phosphates in the LNP was 6. Lipids were dissolved in ethanol and mRNA was dissolved in 50 mM Citrate buffer pH 5.0. The organic and aqueous phases were combined at a flow rate of 12 mL/min on a Precision Nanosystems NanoAssemblr at a ratio of 3:1 (aqueous: organic). The product was immediately diluted to 16.5% ethanol using MilliQ water. The diluted product was dialyzed against a volume of 1×PBS that was 300-fold in excess. The dialyzed product was concentrated using Amicon Ultra 100 kDa centrifuge tubes and sterile filtered. mRNA concentration and encapsulation was assessed by the Invitrogen Ribogreen assay. Step 2: Anti-IgG secondary antibody (RG7) was incubated with various molar excess (5, 10 and 25×) of DBCO-PEG₄-NHS in PBS for the different duration (0.5, 1, 5 or 24 h). The DBCO modified-RG7 was then purified by dialysis in PBS using 10K MWCO overnight and followed by buffer exchange to 1×PBS twice using the Amicon tube (30K MWCO). The DBCO-modified secondary antibody was quantified by BCA assay. The DBCO group per antibody was measured by conjugation of antibody with FAM-Azide 5 isomer and detection of fluorescence against FAM-Azide 5 isomer standard curve. Step 3: The DBCO modified-RG7 was added to LNP at various rations of RG7/azide ratios and incubated for 2 hours at room temperature. Step 4: RG7 conjugated LNPs were separated from unconjugated RG7 using Sepharose CL4B beads on a gel filtration column with PBS as the mobile phase. Fractions containing pure LNPs were identified by fluorescent signal of DiD-loaded in LNPs, and then were pooled together. mRNA concentration and encapsulation were assessed by the Invitrogen Ribogreen assay. RG7 concentration was assessed by the Pierce BCA protein assay kit. Step 5: The targeting (primary) antibody was added to RG7-LNPs at various of primary/secondary weight or molar ratios and incubated for 30 min at room temperature. Step 6 (optional): The targeting antibody bound LNPs were separated from unbound targeting antibody using Sepharose CL4B beads on a gel filtration column with PBS as the mobile phase. Fractions containing pure LNPs were identified by fluorescent signal of DiD-loaded in LNPs, and then were pooled together. mRNA concentration and encapsulation were assessed by the Invitrogen Ribogreen assay.

mCherry mRNA was formulated in LNPs containing a cationic ionizable lipid LIP003, cholesterol, DSPC, PEG-DMG, azide-PEG-DSPE, secondary antibody RG7 and primary antibody (CD19 mAb or isotype IgG) according to the following molar ratios:

| | | | | | | | | | |
|---|---|---|---|---|---|---|---|---|---|
| | | | | | | | | | Purification |
| | LIP003 | Cholesterol | DSPC | PEG-DMG | Azide-PEG-DSPE | RG7/azide | Primary | Primary/RG7 | Post Primary |
| Formulation | (mol %) | (mol %) | mol %) | (mol %) | (mol %) | molar ratio | Antibody | Molar Ratio | Antibody Binding |

| | | | | | | | | |
|---|---|---|---|---|---|---|---|---|
| K347 | 50 | 27.5 | 22 | 0 | 0.5 | 0.1 | CD19 | |
| K348 | 50 | 27.5 | 22 | 0 | 0.5 | 0.05 | CD19 | |
| K349 | 50 | 27.5 | 22 | 0 | 0.5 | 0.01 | CD19 | |
| K351 | 50 | 27.5 | 22 | 0 | 0.5 | 0.26 | CD19 | |
| K352 | 50 | 27.5 | 22 | 0 | 0.5 | 0.13 | CD19 | |
| K353 | 50 | 27.5 | 22 | 0 | 0.5 | 0.03 | CD19 | |
| K354 | 50 | 27.5 | 22 | 0 | 0.5 | 0.14 | CD19 | |
| K355 | 50 | 27.5 | 22 | 0 | 0.5 | 0.07 | CD19 | |
| K356 | 50 | 27.5 | 22 | 0 | 0.5 | 0.01 | CD19 | |
| K357 | 50 | 27.5 | 22 | 0 | 0.5 | 0.26 | CD19 | 0.5 |
| K358 | 50 | 27.5 | 22 | 0 | 0.5 | 0.13 | CD19 | 0.5 |

### Results

As shown in FIG. 28A, the CD19-LNPs made by SATA method (K296, K298 and K300) demonstrated B cell targeting efficiency in vitro in A20 cells but to a less extent than that of the DTT method (K256). The optimal primary/secondary antibody molar ratio of 0.5:1 (K305) was selected for in vivo study. K306 (an additional washing step applied to K305) was also evaluated in vivo. In FIG. 28 B and C, K305 exhibited similar spleen and bone marrow B cell targeting efficiency to its DTT counterpart (K303). K306 with an additional purification step didn't improve B cell targeting efficiency.

As shown in FIG. 29A, the CD19-LNPs made by DBCO/azide method (K347, K348, K349, K351, K352, K353, K354, K355, and K356) demonstrated B cell targeting efficiency in vitro in A20 cells but to a less extent than that of the DTT method (K256-CD19). The optimal primary/secondary antibody molar ratio of 0.5:1 for both K357 and 358 was selected for in vivo study. In FIG. 29B, CD19-LNPs made by DBCO/azide method (K357 and K358) exhibited B cell targeting efficiency but were lower to their DTT counterpart (K258).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A nucleic acid lipid particle composition comprising:
a) a nucleic acid; and
b) lipid components including:

| Component | Range |
|---|---|
| Cationic Ionizable Lipid | 0-60% |
| pKa < 7 | |
| Cationic Ionizable Lipid | 0-80% |
| pKa > 7 | |
| Sterol | 0-40% |
| Helper Lipid | 0-20% |
| PEGylated Lipid | 0-25% |
| PEG-Ligand Lipid | 0-25% |

2. The nucleic acid lipid particle of claim 1, wherein the helper lipid is a phospholipid.

3. A nucleic acid lipid particle composition comprising:
a) a nucleic acid; and
b) lipid components including:

| Component | Range |
|---|---|
| Cationic Ionizable | 30-60% |
| Lipid pKa < 7 | |
| Sterol | 20-40% |
| Helper Lipid | 10-30% |
| PEGylated Lipid | 0-5% |
| PEG-Ligand | 0-5% |

4. The nucleic acid lipid particle of claim 3, wherein the helper lipid is DSPC.

5. The nucleic acid lipid particle of claim 3, wherein the PEGylated lipid is selected from the group consisting of PEG-DMG, PEG-DSPE, Maleimide-PEG-DSPE, Azide-PEG-DSPE and DBCO-PEG-DSPE.

6. The nucleic acid lipid particle of claim 3, wherein the PEG-Ligand is FA-PEG-DSPE

7. The nucleic acid lipid particle of claim 1 or 3, wherein the particle is surface functionalized by a targeting entity

8. The nucleic acid lipid particle of claim 7, wherein the targeting entity comprises an antibody or antibody fragment.

9. The nucleic acid lipid particle of claim 8, wherein the targeting entity comprises a primary antibody or antibody fragment and a secondary antibody or antibody fragment.

10. The nucleic acid lipid particle of claim 9, wherein the secondary antibody or antibody fragment is an anti-IgG antibody or antibody fragment, and the primary antibody or antibody fragment is an anti-CD19 antibody or antibody fragment.

11. The nucleic acid lipid particle of any one of claims 7-10, wherein the targeting entity is covalently linked to the surface of the nucleic acid lipid particle by DTT reduction, SATA thio conjugation or DBCO conjugation.

12. The nucleic acid lipid particle of claim 7-11, wherein the targeting entity is covalently linked to the surface of the nucleic acid lipid particle by maleimide/thio conjugation or DBCO/Azide conjugation.

13. The nucleic acid lipid particle of claim 12, wherein the molar ratio of secondary antibody to maleimide or Azide is 0.01 to 1.

14. The nucleic acid lipid particle of claim 9, wherein the molar ratio of primary to secondary antibody is 0.001 to 1.

15. The nucleic acid lipid particle of claim 13, wherein the molar ratio of secondary antibody to maleimide or Azide is 0.05 to 0.5.

16. The nucleic acid lipid particle of claim 14, wherein the molar ratio of primary to secondary antibody is 0.05, 0.1 and 0.5.

17. A method of delivering a nucleic acid to a tissue of a subject, the method comprising:
administering to the subject the nucleic acid lipid particle composition.

18. The method of claim 17, wherein the nucleic acid lipid particle is a composition of any one of claims 1-16.

19. The method of claim 17, wherein the tissue is or comprises lung tissue.

20. The method of claim 19, wherein the nucleic acid lipid particle is a composition selected from the group consisting of:

| Formulation | Composition |
|---|---|
| K056 | 50 mol % DOTAP, 25 mol % LIP001; |
| | 18.75% cholesterol; 5 mol % DSPC; 0.75 |
| | mol % PEG-DMG, 0.5% PEG-ligand |
| K057 | 50 mol % DOTAP, 25 mol % LIP001; |
| | 19.15% cholesterol; 5 mol % DSPC; 0.75 |
| | mol % PEG-DMG, 0.1% PEG-ligand |
| K058 | 50 mol % DOTAP, 25 mol % LIP001; |
| | 19.2% cholesterol; 5 mol % DSPC; 0.75 |
| | mol % PEG-DMG, 0.05% PEG-ligand |
| K099 | 50 mol % DOTAP, 25 mol % LIP003; |
| | 18.5% cholesterol; 5 mol % DSPC; 0.5 |
| | mol % PEG-DMG, 1% PEG-ligand |
| K100 | 48.2 mol % DOTAP, 24.1 mol % LIP003; |
| | 17.8% cholesterol; 4.8 mol % DSPC; 4 |
| | mol % PEG-DMG, 1% PEG-ligand |
| K101 | 45.7 mol % DOTAP, 22.8 mol % LIP003; |
| | 16.9% cholesterol; 4.6 mol % DSPC; 9 |
| | mol % PEG-DMG, 1% PEG-ligand |
| K102 | 43.1 mol % DOTAP, 21.6 mol % LIP003; |
| | 16% cholesterol; 4.3 mol % DSPC; 14 |
| | mol % PEG-DMG, 1% PEG-ligand |
| K261 | 50 mol % LIP019; 25 mol % LIP003; |
| | 19.25% cholesterol; 5 mol % DSPC; 0.75 |
| | mol % PEG-DMG. |
| K263 | 50 mol % LIP021; 25 mol % LIP003; |
| | 19.25% cholesterol; 5 mol % DSPC; 0.75 |
| | mol % PEG-DMG |
| K269 | 50 mol % LIP021; 25 mol % LIP001; |
| | 19.25% cholesterol; 5 mol % DSPC; 0.75 |
| | mol % PEG-DMG |

21. The method of claim 17, wherein the tissue is extrahepatic.

22. The method of claim 21, wherein the nucleic acid lipid particle is a composition selected from the group consisting of:

| Formulation | Composition |
|---|---|
| K029 | 50 mol % LIP002; 27.5% cholesterol; 22 |
| | mol % DSPC; 0.5 mol % PEG-DMG. |
| K030 | 50 mol % LIP003; 27.5% cholesterol; 22 |
| | mol % DSPC; 0.5 mol % PEG-DMG |
| K108 | 50 mol % LIP003; 27.5% cholesterol; 22 |
| | mol % DSPC; 0.5 mol % PEG-DSPE |
| K111 | 50 mol % LIP003; 25% cholesterol; 22 |
| | mol % DSPC; 3 mol % FA-PEG-DSPE |
| K051 | 46.8 mol % LIP003; 36.8% cholesterol; 15.9 |
| | mol % DSPC; 0.5 mol % PEG-DMG |
| K052 | 46.8 mol % LIP003; 24.9% cholesterol; 27.9 |
| | mol % DSPC; 0.5 mol % PEG-DMG |
| K053 | 34.8 mol % LIP003; 36.8% cholesterol; 27.9 |
| | mol % DSPC; 0.5 mol % PEG-DMG |
| K054 | 42.8 mol % LIP003; 32.8% cholesterol; 23.9 |
| | mol % DSPC; 0.5 mol % PEG-DMG |

23. The method of claim 17, 18, 21, or 22, wherein the tissue is or comprises a tumor.

24. The method of claim 17, 18, 21, or 22, wherein the tissue is or comprises a B cell.

25. The method of claim 17, wherein the tissue is or comprises a B cell and the nucleic acid lipid particle is a composition selected from the group consisting of:

| Formulation | Composition |
|---|---|
| K258 (DTT) | 50 mol % LIP003; 27.5% cholesterol; 22 |
| | mol % DSPC; 0.5 mol % Maleimide-PEG- |
| | DSPE, RG7/Maleimide molar ratio = 0.32/1, |
| | CD19 antibody/RG7 weight ratio = 0.1/1 |
| K259 (DTT) | 50 mol % LIP003; 27.5% cholesterol; 22 |
| | mol % DSPC; 0.5 mol % Maleimide-PEG- |
| | DSPE, RG7/Maleimide molar ratio = 0.32/1, |
| | CD19 antibody/RG7 weight ratio = 1/1 |
| K305 (DTT) | 50 mol % LIP003; 27.5% cholesterol; 22 |
| | mol % DSPC; 0.5 mol % Maleimide-PEG- |
| | DSPE, RG7/Maleimide molar ratio = 0.32/1, |
| | CD19 antibody/RG7 molar ratio-0.5/1 |
| K306 (DTT) | 50 mol % LIP003; 27.5% cholesterol; 22 |
| | mol % DSPC; 0.5 mol % Maleimide-PEG- |
| | DSPE, RG7/Maleimide molar ratio = 0.32/1, |
| | CD19 antibody/RG7 molar ratio-0.5/1 |
| K361 (DTT) | 50 mol % LIP003; 27.5% cholesterol; 22 |
| | mol % DSPC; 0.5 mol % Maleimide-PEG- |
| | DSPE, RG7/Maleimide molar ratio = 0.16/1, |
| | CD19 antibody/RG7 molar ratio = 0.1/1 |
| K362 (DTT) | 50 mol % LIP003; 27.5% cholesterol; 22 |
| | mol % DSPC; 0.5 mol % Maleimide-PEG- |
| | DSPE, RG7/Maleimide molar ratio = 0.06/1, |
| | CD19 antibody/RG7 molar ratio = 0.5/1 |
| K305 (SATA) | 50 mol % LIP003; 27.5% cholesterol; 22 |
| | mol % DSPC; 0.5 mol % Maleimide-PEG- |
| | DSPE, RG7/Maleimide molar ratio = 0.02/1, |
| | CD19 antibody/RG7 molar ratio-0.5/1 |
| K306 (SATA) | 50 mol % LIP003; 27.5% cholesterol; 22 |
| | mol % DSPC; 0.5 mol % Maleimide-PEG- |
| | DSPE, RG7/Maleimide molar ratio = 0.02/1, |
| | CD19 antibody/RG7 molar ratio = 0.5/1 |
| K357 (DBCO/azide) | 50 mol % LIP003; 27.5% cholesterol; 22 |
| | mol % DSPC; 0.5 mol % Maleimide-PEG- |
| | DSPE, RG7/Maleimide molar ratio-0.26/1, |
| | CD19 antibody/RG7 molar ratio = 0.5/1 |
| K358 (DBCO/Azide) | 50 mol % LIP003; 27.5% cholesterol; 22 |
| | mol % DSPC; 0.5 mol % Maleimide-PEG- |
| | DSPE, RG7/Maleimide molar ratio = 0.13/1, |
| | CD19 antibody/RG7 molar ratio = 0.5/1 |

26. A method of treating a subject suffering from a disease, the method comprising
administering to the subject a nucleic acid lipid particle composition.

27. The method of claim 26, wherein the method comprises administering to the subject a nucleic acid lipid particle composition of any one of claims 1-16.

28. The method of claim 26, wherein the method comprises administering to the subject a nucleic acid lipid particle composition selected from the group consisting of:

| Formulation | Composition |
|---|---|
| K056 | 50 mol % DOTAP, 25 mol % LIP001; |
| | 18.75% cholesterol; 5 mol % DSPC; 0.75 |
| | mol % PEG-DMG, 0.5% PEG-ligand |
| K057 | 50 mol % DOTAP, 25 mol % LIP001; |
| | 19.15% cholesterol; 5 mol % DSPC; 0.75 |
| | mol % PEG-DMG, 0.1% PEG-ligand |
| K058 | 50 mol % DOTAP, 25 mol % LIP001; |
| | 19.2% cholesterol; 5 mol % DSPC; 0.75 |
| | mol % PEG-DMG, 0.05% PEG-ligand |
| K099: | 50 mol % DOTAP, 25 mol % LIP003; |
| | 18.5% cholesterol; 5 mol % DSPC; 0.5 |
| | mol % PEG-DMG, 1% PEG-ligand |
| K100 | 48.2 mol % DOTAP, 24.1 mol % LIP003; |
| | 17.8% cholesterol; 4.8 mol % DSPC; 4 |
| | mol % PEG-DMG, 1% PEG-ligand |
| K101 | 45.7 mol % DOTAP, 22.8 mol % LIP003; |
| | 16.9% cholesterol; 4.6 mol % DSPC; 9 |
| | mol % PEG-DMG, 1% PEG-ligand |
| K102 | 43.1 mol % DOTAP, 21.6 mol % LIP003; |
| | 16% cholesterol; 4.3 mol % DSPC; 14 |
| | mol % PEG-DMG, 1% PEG-ligand |
| K261 | 50 mol % LIP019; 25 mol % LIP003; |
| | 19.25% cholesterol; 5 mol % DSPC; 0.75 |
| | mol % PEG-DMG. |
| K263 | 50 mol % LIP021; 25 mol % LIP003; |
| | 19.25% cholesterol; 5 mol % DSPC; 0.75 |
| | mol % PEG-DMG |
| K269 | 50 mol % LIP021; 25 mol % LIP001; |
| | 19.25% cholesterol; 5 mol % DSPC; 0.75 |
| | mol % PEG-DMG |
| K029 | 50 mol % LIP002; 27.5% cholesterol; 22 |
| | mol % DSPC; 0.5 mol % PEG-DMG. |
| K030 | 50 mol % LIP003; 27.5% cholesterol; 22 |
| | mol % DSPC; 0.5 mol % PEG-DMG |
| K108 | 50 mol % LIP003; 27.5% cholesterol; 22 |
| | mol % DSPC; 0.5 mol % PEG-DSPE |
| K111 | 50 mol % LIP003; 25% cholesterol; 22 |
| | mol % DSPC; 3 mol % FA-PEG-DSPE |
| K051 | 46.8 mol % LIP003; 36.8% cholesterol; 15.9 |
| | mol % DSPC; 0.5 mol % PEG-DMG |
| K052 | 46.8 mol % LIP003; 24.9% cholesterol; 27.9 |
| | mol % DSPC; 0.5 mol % PEG-DMG |
| K053 | 34.8 mol % LIP003; 36.8% cholesterol; 27.9 |
| | mol % DSPC; 0.5 mol % PEG-DMG |
| K054 | 42.8 mol % LIP003; 32.8% cholesterol; 23.9 |
| | mol % DSPC; 0.5 mol % PEG-DMG |

29. The method of any one of claims 26-28, wherein the disease is a cancer, a lung disease, a disease effecting a B-Cell.

30. A method of manufacturing a nucleic acid lipid particle comprising combining a nucleic acid with lipid components including:

| Component | Range |
|---|---|
| Cationic Ionizable | 0-60% |
| Lipid < 7 pKa | |
| Cationic Ionizable | 0-80% |
| Lipid > 7 pKa | |
| Sterol | 0-40% |
| Helper Lipid | 0-30% |
| PEGylated Lipid | 0-25% |
| PEG-Ligand Lipid | 0-25% |

31. A method of evaluating LNP formulations for delivery to the lung, the method comprising steps of:
formulating a nucleic acid payload in an LNP formulation as set forth below: K020:
50 mol % LIP005; 25 mol % LIP001; 19.25% cholesterol; 5 mol % DSPC; 0.75 mol % PEG-DMG
formulating the nucleic acid payload in at least one test LNP formulation; and
determining payload expression level in lung or ratio of liver: lung expression for the at least one test LNP formulation relative to LNP4.

32. The nucleic acid of claim 1 or 2; the method of claim 17, 26, or 30 wherein the cationic ionizable lipid pKa<7 is selected from Table 2.

33. The nucleic acid of claim 1 or 2; the method of claim 17, 26, or 30, wherein the cationic ionizable lipid pKa>7 is selected from Table 1.

34. The nucleic acid lipid particle of claim 1 or 2, the method of claim 17, 26, or 30 wherein the Cationic Ionizable Lipid with pKa>7 is one of the following LIP021, LIP019, LIP022, LIP020, or LIP005.

35. The nucleic acid lipid particle of claim 1 or 2, the method of claim 17, 26, or 30 wherein the Cationic Ionizable Lipid with pKa<7 is one of the following LIP002, LIP001, LIP003, LIP007, LIPO18, LIPO62, LIPO10 and LIP011.

36. The nucleic acid lipid particle of any of the preceding claims, wherein the nucleic acid is an RNA

37. The nucleic acid lipid particle of claim 36, wherein the RNA is or comprises mRNA, antisense RNA, siRNA, shRNA, miRNA, gRNA, or a combination thereof.

38. A method of functionalizing a nucleic acid lipid particle, the method comprising conjugating a targeting entity to the surface of the nucleic acid lipid particle.

39. The method of claim 38, wherein the conjugation comprises linking the targeting entity to the surface of the nucleic acid lipid particle by DTT reduction, SATA thio conjugation or DBCO conjugation.

40. The method of claim 38, wherein the conjugation comprises linking the targeting entity to the surface of the nucleic acid lipid particle by maleimide/thio conjugation or DBCO/Azide conjugation.

41. The method of claim 38, wherein the targeting entity comprises an antibody or antibody fragment.

42. The method of claim 41, wherein the targeting entity comprises a primary antibody or antibody fragment and a second antibody or antibody fragment.

43. The method of claim 42, wherein the secondary antibody or fragment is an anti-IgG antibody or fragment, and the primary antibody or fragment is an anti-CD19 antibody or fragment.

44. The method of claim 40, wherein the molar ratio of secondary antibody or fragment to maleimide or Azide is 0.01 to 1.

45. The method of claim 42, wherein the molar ratio of primary antibody or fragment to secondary antibody or fragment is 0.001 to 1.

46. The method of claim 44, wherein the molar ratio of secondary antibody to maleimide or Azide is 0.05 to 0.5.

47. The method of claim 44, wherein the molar ratio of primary antibody or fragment to secondary antibody or fragment is 0.05, 0.1 and 0.5.
